(19) United States
(12) Patent Application Publication
(10) Pub. No.: US 2024/0182407 A1
(43) Pub. Date: Jun. 6, 2024

(54) QUINOLINE COMPOUND SALT OR CRYSTAL FORM, PREPARATION METHOD THEREFOR, AND APPLICATION THEREOF

(71) Applicant: Nanjing Zenshine Pharmaceuticals Co., Ltd., Nanjing (CN)

(72) Inventor: Xiaolin Hao, Foster City, CA (US)

(21) Appl. No.: 18/549,596

(22) PCT Filed: Mar. 8, 2022
(86) PCT No.: PCT/CN2022/079640

§ 371 Date: Sep. 8, 2023

(30) Foreign Application Priority Data
Mar. 9, 2021 (CN) 202110255314.6
Mar. 9, 2021 (CN) 202110257056.5

(51) Int. Cl. C07C 309/73; A61K 31/255; A61K 31/506; C07C 303/26; C07C 303/44; C07D 401/14

(52) U.S. Cl. CPC C07C 309/73; A61K 31/255; A61K 31/506; C07C 303/26; C07C 303/44; C07D 401/14; C07B 2200/13

(57) ABSTRACT

The present invention relates to the field of biomedicine; provided are a quinoline compound salt or a crystal form thereof, a preparation method therefor and an application thereof. The quinoline compound is as shown in Formula (I), and the provided salt may be used as an inhibitor of phosphoinositide 3-kinase for the treatment of diseases related to phosphoinositide 3-kinase. More particularly, the compound of Formula (I) may be 2,4-diamino-6-[1-(7-fluoro-2-pyridin-2-yl-quinolin-3-yl)-ethylamino]-pyrimidine-5-carbonitrile (Compound A), the structure thereof being as shown below. Crystal form I of p-toluenesulfonate of the compound may be used as an inhibitor of phosphoinositide 3-kinase for the treatment of diseases related to phosphoinositide 3-kinase.

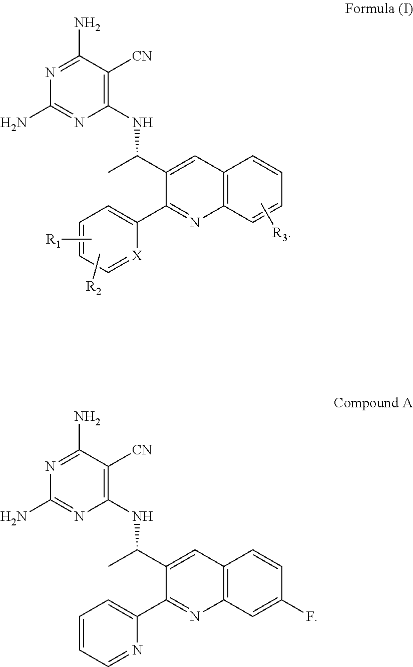

### TECHNICAL FIELD

The present invention relates to the field of biomedicine, in particular to a quinoline compound salt or a crystal form thereof, a preparation method therefor and an application thereof.

### BACKGROUND ART

Phosphoinositide 3-kinases (PI3Ks) belong to a large family of lipid signaling kinases. Among them, class I PI3Ks (including PI3Kα, PI3Kβ, PI3Kγ and PI3Kδ) belong to the family of bispecific lipid and protein kinases. PI3Ks inherently have serine/threonine (Ser/Thr) kinase activity and can phosphorylate phosphatidylinositol 4,5-diphosphate (PIP₂), thus producing phosphatidylinositol-3,4,5-triphosphate (PIP₃). PIP₃ plays a key role in cell survival, signaling, control of transmembrane transport, and other functions and is involved in the regulation of a variety of cell functions such as cell proliferation, differentiation, apoptosis, and glucose transport (Di Paolo, G. et al., Nature, 2006,443,651; Parker, P. J. et al., Biochem. Soc. Trans. 2004, 32, 893; Hawkins, P. T. et al., Biochem. Soc. Trans. 2006, 34, 647; and Schaeffer, E. M. et al., Curr. Opin. Immnunol. 2000, 12, 2822). If the regulation mechanism is abnormal, many diseases such as cancer, inflammation and autoimmune diseases will be caused.

PI3Ks can be divided into three categories with different structures and functions. Among them, class I PI3Ks are the most widely studied. Class I PI3Ks consist of four kinases, which can be further divided into two subclasses. Among them, subclass 1A PI3Ks consist of three closely related kinases PI3Kα, PI3Kβ and PI3Kδ, all of which exist in the form of heterodimers and are composed of catalytic subunits (p110α, p110β or p110δ) and different kinds of regulatory subunits. Subclass 1A PI3Ks typically respond to signaling pathways via receptor tyrosine kinases (RTKs). Subclass 1B consists of PI3Kγ alone, which mainly responds to signaling pathways via G protein-coupled receptors (GPCRs). Similar to the structure of subclass 1A PI3Ks, PI3Kγ consists of catalytic subunit p110γ and one of two different regulatory subunits. PI3Kα and PI3Kβ are widely expressed in various tissues and organ types. PI3Kγ mainly exists in white blood cells and is also found in skeletal muscle, liver, pancreas and heart (Cantly, C. Science 2002, 1655). The expression pattern of PI3Kδ is limited by spleen, thymus and peripheral blood leukocytes (Knight, Z. et al., Cell 2006, 125, 733).

PI3Kδ, one of the four kinases of class I PI3Ks, is also an important member of PI3K-AKT-mTOR signaling pathway and is also considered as the main participant in the function of adaptive immune system in vivo. This pathway is critical for tumor growth, and tumor cells rely on this pathway to maintain growth, metastasis and diffusion. Studies have shown that PI3Kδ plays an important role in regulating adaptive immune system cells (B cells, and T cells to a lesser extent) and innate immune system cells (neutrophils, mast cells and macrophages) and is a potentially effective therapeutic target for many immune diseases.

The latest research results have indicated that if p110δ in mice is inactivated, many cancers, including non-hematological solid tumors, can be prevented. In addition, p110δ inactivation in regulatory T cells (Tregs) results in the release of CD8+ cytotoxic T cells and the induction of tumor regression. Therefore, p110δ inhibitors can destroy tumor-induced immune tolerance and have potential wide applications in clinical treatment of tumors (Ali, et al., Nature: 2014, 510, 407-411).

In July 2014, the first PI3Kδ inhibitor Idelalisib was approved by FDA and EMA to treat various types of leukemia. To date, three new drugs, Idelalisib, Copanlisib and Duvelisib, which have inhibitory effect on PI3K8, have been approved in the United States one after another. PI3Kδ has gradually entered people's field of vision and attracted the attention of new drug developers.

The global research and development of new drugs against this target is in an active period, and PI3Kδ inhibitors such as Parsaclisib, HMPL-689, Copanlisib and CDZ173 are also in preclinical or clinical trials.

Although a number of PI3Kδ inhibitors have been put on the market or are under research, there still remains a huge demand for PI3Kδ inhibitors with better clinical efficacy and less toxic and side effects. By improving the stability of PI3Kδ inhibitors in vivo, overcoming the CYP enzyme inhibition or induction tendency and combining with other anti-cancer intervention (such as emerging immunotherapies) therapies, the clinical potential of PI3Kδ inhibitors in the field of the treatment of malignant tumors can be further released. As more companies put research and development into the clinical development directed at this target, safer and more effective PI3Kδ inhibitors will be developed and applied to the treatment of clinical patients in the future.

### SUMMARY OF THE INVENTION

The present invention provides a novel salt of a quinoline compound, i.e., a PI3K subtype inhibitor with significantly improved properties.

In a first aspect of the present invention, there is provided a salt which is an organic acid salt or inorganic acid salt of a compound shown in Formula (I), wherein the organic acid comprises at least one selected from toluenesulfonic acid, benzenesulfonic acid, methanesulfonic acid, ethanesulfonic acid and maleic acid; the inorganic acid comprises at least one selected from hydrochloric acid, hydrobromic acid and sulfuric acid; and

the compound shown in Formula (I) is

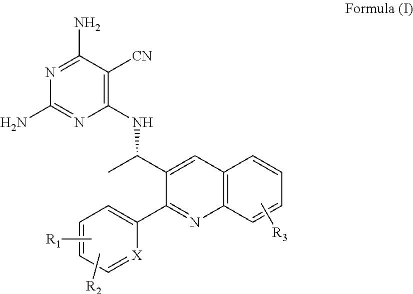

in which X is selected from N or CH;

each of R₁ and R₂ is independently selected from H, F, and SO₂Me, and

R₃ is selected from F and Cl.

In a second aspect of the present invention, there is provided a p-toluenesulfonate of Compound A, wherein Compound A is:

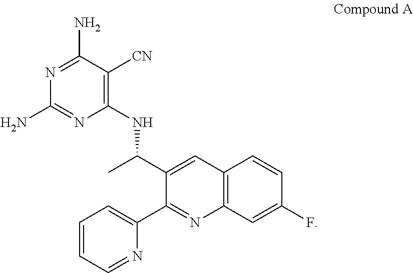 [SMILES: C[C@H](Nc1nc(N)nc(N)c1C#N)c1cc2ccc(F)cc2nc1-c1ccccn1]

In a third aspect of the present invention, there is provided a pharmaceutical composition is provided, which comprises the above-mentioned organic acid salt or inorganic acid salt of the compound shown in Formula (I) or the above-mentioned p-toluenesulfonate of Compound A, and a pharmaceutically acceptable carrier.

In a fourth aspect of the present invention, there is provided a method for preparing the above-mentioned organic acid salt or inorganic acid salt of the compound shown in Formula (I), comprising: reacting a compound shown in Formula (I) with an organic acid or inorganic acid to form the salt.

In a fifth aspect of the present invention, there is provided a method for preparing the above-mentioned compound shown in Formula (I).

In a sixth aspect of the present invention, there is provided a method for selectively inhibiting the growth or proliferation of cells containing phosphoinositide 3-kinase in vitro, comprising:

bringing the cells into contact with an effective amount of the above-mentioned organic acid salt or inorganic acid salt of the compound shown in Formula (I) or the above-mentioned p-toluenesulfonate of Compound A or the above-mentioned pharmaceutical composition.

In a seventh aspect of the present invention, there is provided a method for preventing or treating a disease related to phosphoinositide 3-kinase, comprising administering an effective amount of the above-mentioned organic acid salt or inorganic acid salt of the compound shown in Formula (I) or the above-mentioned p-toluenesulfonate of Compound A or the above-mentioned pharmaceutical composition to a subject.

In an eighth aspect of the present invention, there is provided the use of the above-mentioned organic acid salt or inorganic acid salt of the compound shown in Formula (I) or the above-mentioned p-toluenesulfonate of Compound A or the above-mentioned pharmaceutical composition in the preparation of a medicament for preventing or treating a disease related to phosphoinositide 3-kinase.

In a ninth aspect of the present invention, there is provided the above-mentioned organic acid salt or inorganic acid salt of the compound shown in Formula (I) or the above-mentioned p-toluenesulfonate of Compound A or the above-mentioned pharmaceutical composition, for preventing or treating a disease related to phosphoinositide 3-kinase.

The present invention further provides a novel crystal form of a salt of a quinoline compound, i.e., a PI3K subtype inhibitor with significantly improved properties.

In a tenth aspect of the present invention, there is provided a crystal form of p-toluenesulfonate of Compound A, which is crystal form I,

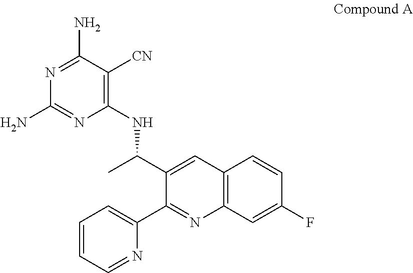 [SMILES: C[C@H](Nc1nc(N)nc(N)c1C#N)c1cc2ccc(F)cc2nc1-c1ccccn1]

the crystal form I has XRPD characteristic peaks at 2θ values of 4.9°±0.2°, 7.6°±0.2°, 12.2°±0.2°, 14.8°±0.2° and 15.4°±0.2°.

In an eleventh aspect of the present invention, there is provided a pharmaceutical composition, which comprises the above-mentioned crystal form and a pharmaceutically acceptable carrier.

In a twelfth aspect of the present invention, there is provided the use of the above-mentioned crystal form or the above-mentioned pharmaceutical composition in the preparation of a medicament for preventing or treating a disease related to phosphoinositide 3-kinase.

In a thirteenth aspect of the present invention, there is provided a method for selectively inhibiting the growth or proliferation of cells containing phosphoinositide 3-kinase in vitro, comprising:

bringing the cells into contact with an effective amount of the above-mentioned crystal form or the above-mentioned pharmaceutical composition.

In a fourteenth aspect of the present invention, there is provided a method for preventing or treating a disease related to phosphoinositide 3-kinase, comprising administering an effective amount of the above-mentioned crystal form or the above-mentioned pharmaceutical composition to a subject.

In a fifteenth aspect of the present invention, there is provided the above-mentioned crystal form or the above-mentioned pharmaceutical composition, for preventing or treating a disease related to phosphoinositide 3-kinase.

### BRIEF DESCRIPTION OF THE DRAWINGS

FIG. 1 is the ¹H NMR spectrum results of some samples in a 96-well plate provided according to an embodiment of the present invention.

FIG. 2 is the ¹H NMR spectrum results of some samples in a 96-well plate provided according to an embodiment of the present invention.

FIG. 3 is the XRPD pattern results of some samples in row 4 of a 96-well plate provided according to an embodiment of the present invention.

FIG. 4 is the XRPD pattern results of some samples in column E of a 96-well plate provided according to an embodiment of the present invention.

FIG. 5 is the XRPD pattern results of p-toluenesulfonates provided according to an embodiment of the present invention.

FIG. 6 is the PLM (polarized light microscopy) pattern results of a p-toluenesulfonate (Sample 3) provided according to an embodiment of the present invention.

FIG. 7 is the TGA-DSC pattern results of a p-toluenesulfonate (Sample 3) provided according to an embodiment of the present invention.

FIG. 8 is the DVS pattern results of a p-toluenesulfonate (Sample 3) provided according to an embodiment of the present invention.

FIG. 9 is the XRPD pattern results of a p-toluenesulfonate (Sample 3) provided according to an embodiment of the present invention after DVS.

FIG. 10 is the XRPD pattern results of Samples 5 and 3 provided according to an embodiment of the present invention.

FIG. 11 is the dynamic water desorption analysis (DVS) pattern results of Sample 3 provided according to an embodiment of the present invention.

FIG. 12 is a histogram of the solubility test results of Compound A and p-toluenesulfonate of Compound A provided according to an embodiment of the present invention.

FIG. 13 is the XRPD pattern results of Sample 8 provided according to an embodiment of the present invention.

FIG. 14 is the TGA-DSC pattern results of Sample 8 provided according to an embodiment of the present invention.

FIG. 15 is the ¹H NMR spectrum results of Sample 8 provided according to an embodiment of the present invention.

FIG. 16 is the XRPD pattern results of Sample 8 provided according to an embodiment of the present invention after high humidity stability determination.

FIG. 17 is the 1H NMR spectrum results of Sample 6 provided according to an embodiment of the present invention.

FIG. 18 is the XRPD pattern results of Sample 6 provided according to an embodiment of the present invention.

FIG. 19 is a diagram showing the results of the anti-tumor effects of various compounds provided according to the embodiment of the present invention on a human-derived lymphoma DoHH-2 cell line subcutaneous transplantation CB17/SCID female immunodeficient mouse model.

### DETAILED DESCRIPTION OF EMBODIMENTS

The embodiments described below by referring to the drawings are exemplary and are intended to explain the present invention, but not to be construed as limiting the present invention.

### Salt of Compound

The present invention provides a salt which is an organic acid salt or inorganic acid salt of a compound shown in Formula (I), wherein the organic acid comprises at least one selected from toluenesulfonic acid, benzenesulfonic acid, methanesulfonic acid, ethanesulfonic acid and maleic acid; the inorganic acid comprises at least one selected from hydrochloric acid, hydrobromic acid and sulfuric acid; and

the compound shown in Formula (I) is

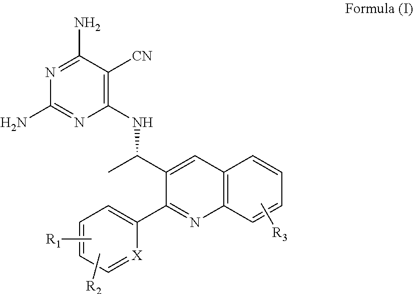

in which X is selected from N or CH;

each of R₁ and R₂ is independently selected from H, F, and SO₂Me, and

R₃ is selected from F and Cl.

By forming the compound shown in Formula (I) into an organic acid salt or inorganic acid salt, a compound salt with high solubility is obtained. The salt easily forms a stable crystal, and the stability of the compound salt is higher than that of the compound itself.

In some embodiments, in the compound shown in Formula (I), R₃ is selected from 7-F, 8-F and 8-Cl. In some embodiments, in the compound shown in Formula (I), X is N, and each of R₁ and R₂ is H. In some embodiments, in the compound shown in Formula (I), X is CH, and R₁ is SO₂Me. In some embodiments, in the compound shown in Formula (I), X is CH, and R₁ is F. In some embodiments, X is CH, R₁ is H, and R₂ is H.

In some embodiments, there is provided an organic acid salt or inorganic acid salt of the compound shown in Formula (I), wherein in the compound shown in Formula (I), X is N, each of R₁ and R₂ is H, and R₃ is 7-F. In some embodiments, there is provided an organic acid salt or inorganic acid salt of the compound shown in Formula (I), wherein in the compound shown in Formula (I), X is C, R₁ is H, R₂ is 2-SO₂Me, and R₃ is 8-F.

It should be noted that each or every variant of R₁ and R₂ can be combined with each X or variant of X as described for the compound of Formula (I), and the compound obtained by this combination also falls within the scope of protection of the present invention. In some embodiments, the organic acid comprises at least one selected from toluenesulfonic acid and methanesulfonic acid.

In some embodiments, the organic acid is p-toluenesulfonic acid, m-toluenesulfonic acid or o-toluenesulfonic acid.

In some embodiments, the present application provides p-toluenesulfonate of Compound A, wherein Compound A is:

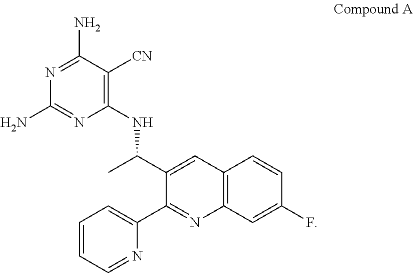 [SMILES: C[C@H](Nc1nc(N)nc(N)c1C#N)c1cc2ccc(F)cc2nc1-c1ccccn1]

The p-toluenesulfonate of Compound A easily forms a stable crystal form, which has a high solubility and exhibits a high stability under high temperature and high humidity conditions.

Crystal Form of p-toluenesulfonate of Compound A

The present invention provides a crystal form of p-toluenesulfonate of Compound A, which is crystal form I,

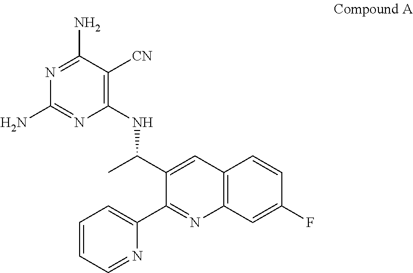 [SMILES: C[C@H](Nc1nc(N)nc(N)c1C#N)c1cc2ccc(F)cc2nc1-c1ccccn1]

The crystal form I has XRPD characteristic peaks at 2θ values of about 4.9°, about 7.6°, about 12.2°, about 14.8°, and about 15.4°.

In the present invention, crystal form I is obtained by crystallizing p-toluenesulfonate of Compound A. Moreover, with the p-toluenesulfonate of Compound A as the starting material, crystal form screening is carried out by means of a variety of methods, such as volatile crystallization, suspension beating, anti-solvent precipitation, cooling crystallization and grinding, and after screening for various crystal forms of p-toluenesulfonate of Compound A, it was found that no new crystal form appeared except crystal form I. Moreover, the prepared crystal form I of p-toluenesulfonate exhibits a high stability under high humidity conditions, and crystal form I is essentially non-hygroscopic under 0-90% RH condition and exhibits an extremely low hygroscopicity.

In some embodiments, crystal form I has XRPD characteristic peaks at 2θ values of 4.9°±0.2°, 7.6°±0.2°, 12.2°±0.2°, 14.8°±0.2° and 15.4°±0.2°.

In some embodiments, crystal form I has XRPD characteristic peaks at 2θ values of 4.9°±0.1°, 7.6°±0.1°, 12.2°±0.1°, 14.8°±0.1° and 15.4°±0.1°.

In some embodiments, crystal form I further has at least one XRPD characteristic peak selected from those at 2θ values of about 9.8°, about 10.3°, about 14.3°, about 14.5°, about 16.3°, about 18.3° and about 19.8°. For example, one, two, three, four, five, six or seven XRPD characteristic peaks selected from those at 2θ values of about 9.8°, about 10.3°, about 14.3°, about 14.5°, about 16.3°, about 18.3° and about 19.8° may be contained.

In some embodiments, crystal form I further has at least one XRPD characteristic peak selected from those at 2θ values of 9.8°±0.2°, 10.3°±0.2°, 14.3°±0.2°, 14.5°±0.2°, 16.3°±0.2°, 18.3°±0.2° and 19.8°±0.2°. For example, one, two, three, four, five, six or seven XRPD characteristic peaks selected from those at 2θ values of 9.8°±0.2°, 10.3°±0.2°, 14.3°±0.2°, 14.5°±0.2°, 16.3°±0.2°, 18.3°±0.2° and 19.8°±0.2° may be contained.

In some embodiments, crystal form I further has at least one XRPD characteristic peak selected from those at 2θ values of 9.8°±0.1°, 10.3°±0.1°, 14.3°±0.1° , 14.5°±0.1°, 16.3°±0.1°, 18.3°±0.1° and 19.8°±0.1°. For example, one, two, three, four, five, six or seven XRPD characteristic peaks selected from those at 2θ values of 9.8°±0.1°, 10.3°±0.1°, 14.3°±0.1°, 14.5°±0.1°, 16.3°±0.1°, 18.3°±0.1° and 19.8°±0.1° may be contained.

In some embodiments, crystal form I has an X-ray powder diffraction pattern essentially as shown in FIG. 13.

In some embodiments, the X-ray powder diffraction data in FIG. 13 are as shown in the following table:

| | Relative |
|---|---|
| 2-theta ° | intensity % |
| 4.876 | 100 |
| 7.631 | 9.3 |
| 8.752 | 2.6 |
| 9.839 | 4.1 |
| 10.33 | 3.9 |
| 12.241 | 13.6 |
| 14.318 | 5.1 |
| 14.542 | 5.6 |
| 14.819 | 11.8 |
| 15.364 | 10.3 |
| 16.346 | 5.1 |
| 17.056 | 1.5 |
| 17.634 | 1.4 |
| 18.32 | 4.9 |
| 18.788 | 1.8 |
| 19.128 | 3.2 |
| 19.816 | 3.9 |
| 20.086 | 2.6 |
| 20.547 | 2 |
| 21.411 | 3 |
| 22.061 | 1.8 |
| 22.488 | 1.7 |
| 23.206 | 2.1 |
| 23.499 | 1.7 |
| 24.089 | 6 |
| 24.702 | 1.8 |
| 25.199 | 1.3 |
| 25.932 | 1.3 |
| 26.338 | 1.6 |
| 27.752 | 1 |
| 28.128 | 1.3 |
| 28.389 | 1.5 |
| 28.945 | 1.7 |
| 29.805 | 1 |
| 33.035 | 0.8 |
| 33.469 | 0.8 |
| 33.826 | 0.8 |
| 35.855 | 0.7 |
| 36.24 | 0.8 |
| 38.043 | 0.7 |
| 38.553 | 0.8 |

In some embodiments, crystal form I has a melting peak at 277° C.-283° C.

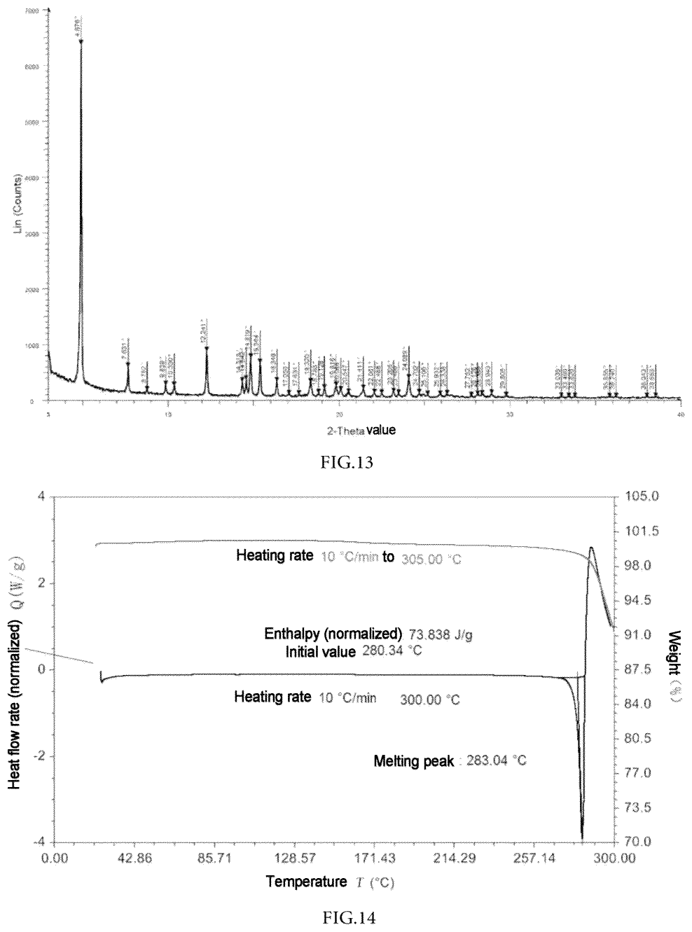

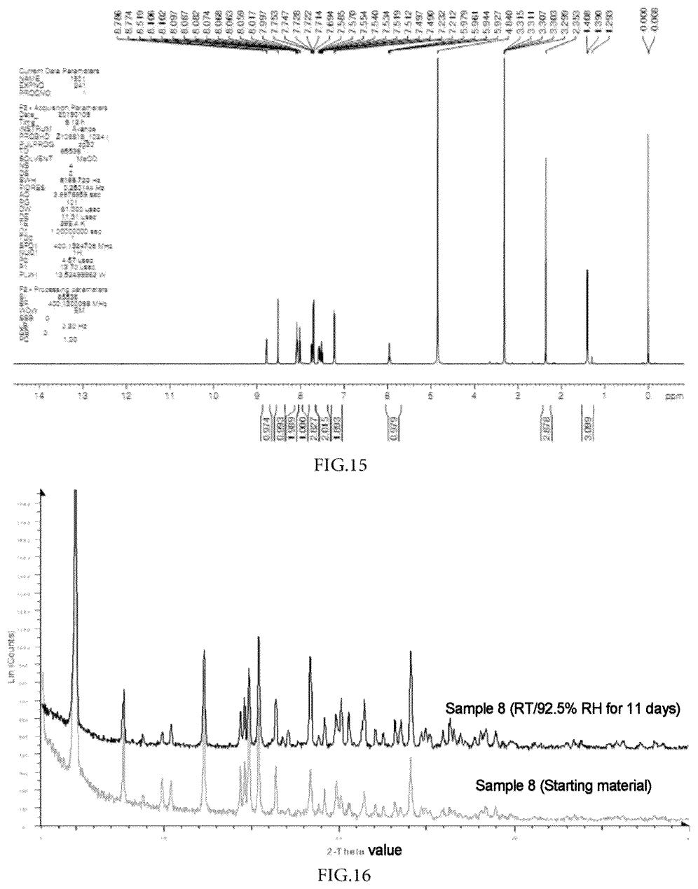

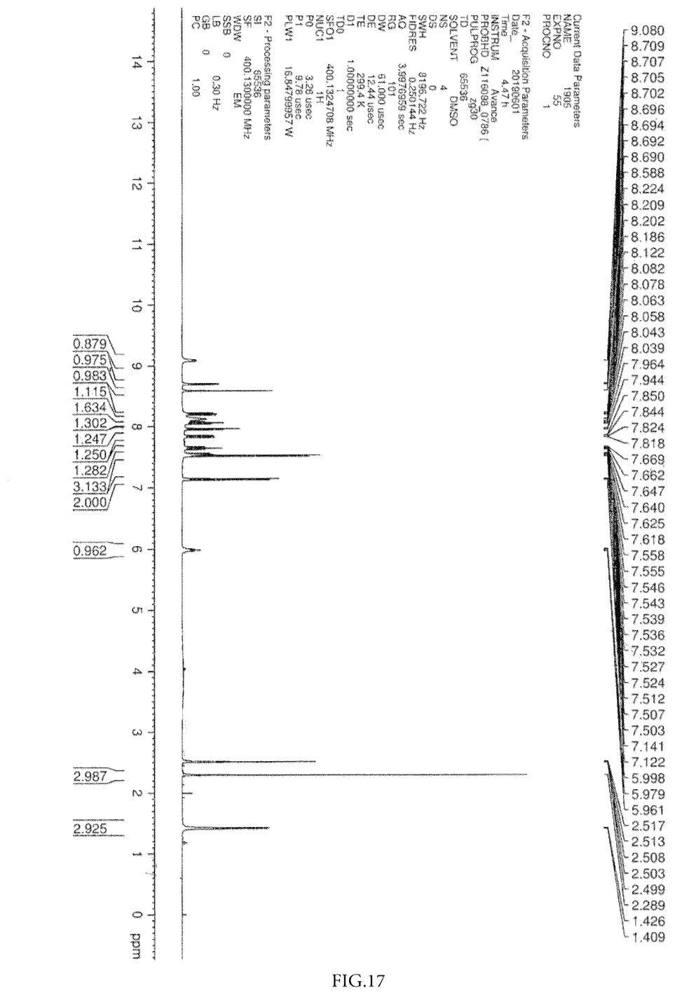

In some embodiments, crystal form I has DSC and TGA thermograms essentially as shown in FIG. 14.

### Pharmaceutical Composition

The present invention further provides a pharmaceutical composition, which comprises the above-mentioned salt or crystal form, such as the above-mentioned p-toluenesulfonate of Compound A or crystal form I thereof, and a pharmaceutically acceptable carrier.

Pharmaceutically acceptable carriers include but are not limited to lactose, dextrose, sucrose, sorbitol, mannitol, starch, Arabic gum, calcium phosphate, alginate, tragacanth, gelatin, calcium silicate, microcrystalline cellulose, polyvinylpyrrolidone, cellulose, water, syrup and methylcellulose. In addition, according to the requirements of different preparations, the provided pharmaceutical composition may also comprise a lubricant, such as talcum powder, magnesium stearate or mineral oil, a wetting agent, an emulsifier, a suspending agent, a preservative such as methyl benzoate and propyl hydroxybenzoate, a sweetener, etc.

### Preparation Method

The present application further provides a method for preparing the above-mentioned organic acid salt or inorganic acid salt of the compound shown in Formula (I), comprising: reacting a compound shown in Formula (I) with an organic acid or inorganic acid to form the salt.

In some embodiments, the method further comprises: preparing a reaction product of a compound shown in Formula (I) and an organic acid or inorganic acid in an organic solvent; and recovering a solid from the reaction product by filtration.

In some embodiments, the reaction is carried out at 20-50 degrees Celsius. In some preferred embodiments, the reaction is carried out at 25-40 degrees Celsius.

In some embodiments, the organic solvent comprises at least one selected from methanol, isopropanol, tetrahydrofuran, 2-methyltetrahydrofuran, acetonitrile, ethanol, methyl tert-butyl ether, acetone and ethyl acetate. In some preferred embodiments, the organic solvent comprises at least one selected from acetone and ethyl acetate.

In some embodiments, the compound shown in Formula (I) can be prepared by the following method:

reacting a compound shown in Formula (II) with a compound shown in Formula (III) to form a compound shown in Formula (IV);

reacting the compound shown in Formula (IV) with an acid to form a compound shown in Formula (V); and

reacting the compound shown in Formula (V) with 2,4-diamino-6-chloropyrimidine-5-carbonitrile to form the compound shown in Formula (I);

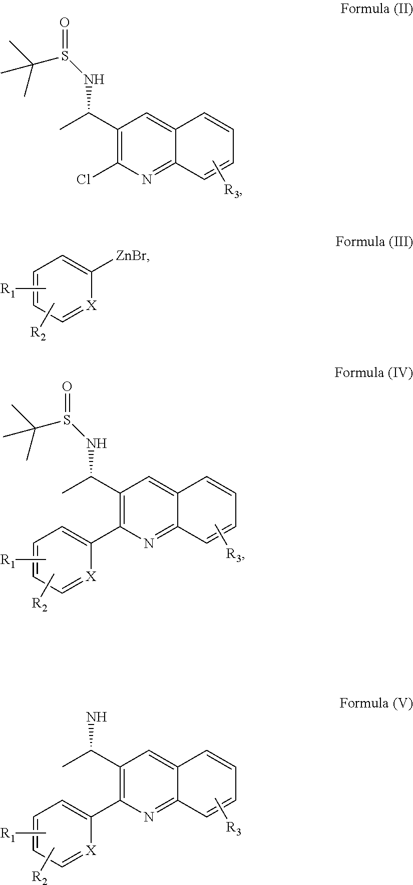

wherein the group R₃ in each of the compounds shown in Formulas (II), (IV) and (V) is the same as the group R₃ in the compound shown in Formula (I);

the groups R₁ and R₂ in the compounds shown in Formulas (III), (IV) and (V) are respectively the same as the groups R₁ and R₂ in the compound shown in Formula (I); and

X in the compounds shown in Formulas (III), (IV) and (V) is selected from N or CH.

In some embodiments, group R₃ in the compounds shown in Formulas (II), (IV) and (V) is F or Cl.

In some embodiments, groups R₁ and R₂ in the compounds shown in Formulas (III), (IV) and (V) are each independently selected from H, F or SO₂Me.

The present invention further provides a method for preparing the above-mentioned p-toluenesulfonate of Compound A, comprising the following steps: reacting Compound A with p-toluenesulfonic acid to form the p-toluenesulfonate of Compound A.

In some embodiments, the method further comprises: preparing a reaction product of Compound A and p-toluenesulfonic acid in an organic solvent; and recovering a solid from the reaction product by filtration.

In some embodiments, the reaction is carried out at 20-50 degrees Celsius. In some preferred embodiments, the reaction is carried out at 25-40 degrees Celsius.

In some embodiments, the organic solvent comprises at least one selected from methanol, isopropanol, tetrahydrofuran, 2-methyltetrahydrofuran, acetonitrile, ethanol, methyl tert-butyl ether, acetone and ethyl acetate. In some preferred embodiments, the organic solvent comprises at least one selected from acetone and ethyl acetate.

In some embodiments, Compound A can be prepared by the following method:
reacting Compound B with Compound C to form Compound D;
reacting Compound D with an acid to form Compound E; and
reacting Compound E with 2,4-diamino-6-chloropyrimidine-5-carbonitrile to form Compound A.

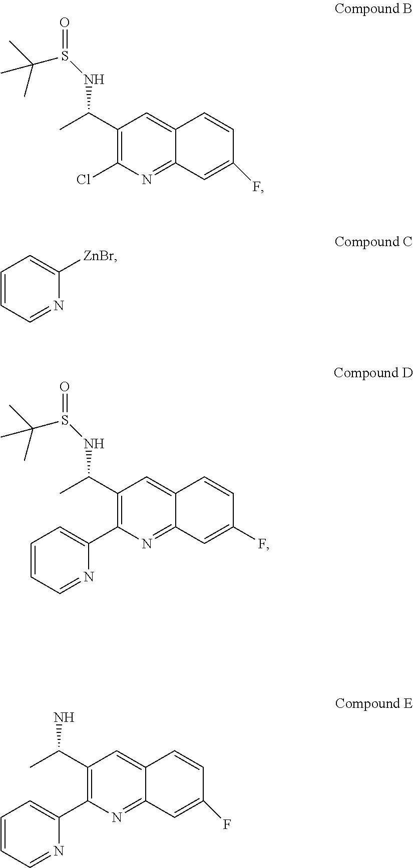

Compounds, the synthesis routes of which are not specifically listed herein, can be prepared by using known organic synthesis techniques and can be synthesized according to any of various possible synthesis routes; or may also be directly purchased. The generated substances can be monitored according to any suitable method known in the art. For example, product formation can be monitored by spectroscopic means, such as nuclear magnetic resonance spectroscopy (e.g., ¹H or ¹³C), infrared spectroscopy or spectrophotometry, or by chromatography, such as high performance liquid chromatography (HPLC) or thin layer chromatography (TLC) or other techniques.

A compound shown in Formula (I) prepared according to the above-mentioned method or Compound A can react with the organic acid or inorganic acid mentioned herein to form a salt of the compound shown in Formula (I) or p-toluenesulfonate of Compound A.

The compounds or salts of the compounds mentioned herein (e.g., p-toluenesulfonate of Compound A) are substantially separated. The term “substantially separated” means that a compound or a salt of the compound can be at least partially or substantially separated from the environment in which it is formed or detected. The term “substantially separated” means that at least about 60 wt %, at least about 70 wt %, at least about 70 wt %, at least about 80 wt %, at least about 90 wt %, at least about 95 wt %, at least about 97 wt %, or at least about 99 wt % of the compound of the present invention or the salt of the compound, Compound A, or crystal form I of p-toluenesulfonate of Compound A can be comprised. Methods for separating the compound or the salt of the compound, Compound A, or crystal form I of p-toluenesulfonate of Compound A are conventional in the art.

### Therapeutic Method and Use

The present application further provides the use of the above-mentioned organic acid salt or inorganic acid salt of the compound shown in Formula (I), or the above-mentioned p-toluenesulfonate of Compound A or crystal form I thereof, or the above-mentioned pharmaceutical composition in the preparation of a medicament for preventing or treating a disease related to phosphoinositide 3-kinase.

In some embodiments, the present application provides a method for selectively inhibiting the growth or proliferation of cells containing phosphoinositide 3-kinase in vitro, comprising:

bringing the cells into contact with an effective amount of the above-mentioned organic acid salt or inorganic acid salt of the compound shown in Formula (I), or the above-mentioned p-toluenesulfonate of Compound A or crystal form I thereof, or the above-mentioned pharmaceutical composition.

The present application further provides a method for preventing or treating a disease related to phosphoinositide 3-kinase, comprising administering an effective amount of the above-mentioned organic acid salt or inorganic acid salt of the compound shown in Formula (I), or the above-mentioned p-toluenesulfonate of Compound A or crystal form I thereof, or the above-mentioned pharmaceutical composition to a subject.

The activity of the phosphoinositide 3-kinase mentioned herein mainly refers to the activity of phosphoinositide 3-kinase 8 (PI3Kδ). The inhibition of the activity of PI3Kδ or the variant thereof refers to the decrease of the activity of PI3Kδ, which is relative to the activity of PI3Kδ in the absence of the salt of the compound shown in Formula (I) or the p-toluenesulfonate of compound A or crystal form I thereof, and is a direct or indirect response in the presence of the salt of the compound shown in Formula (I) or the p-toluenesulfonate of compound A or crystal form I thereof. The salt of the compound shown in Formula (I) or the p-toluenesulfonate of Compound A or crystal form I thereof, as mentioned herein, can also be used for inhibiting the activity of PI3Kγ, and the inhibitory activity exhibited thereon is weaker than on the activity of PI3Kδ.

In some embodiments, the disease related to phosphoinositide 3-kinase as mentioned is a PI3Kδ activity-related disease.

As used herein, the term “treatment” or “prevention” with respect to a disease means to alleviate or prevent one or more biological manifestations of the disease, so as to intervene at one or more points in the biological cascade that causes or is the cause of the disease, thereby alleviating one or more symptoms or effects related to the disease. As mentioned above, the term “treatment” of a disease includes the prevention of the disease, and the term “prevention” should be understood to mean the preventive administration of a drug to significantly reduce the possibility or severity of the disease or a biological manifestation thereof or to delay the onset of such a disease or the biological manifestation thereof.

Those skilled in the art would understand that the following examples are only used to illustrate the present invention and should not be regarded as limiting the scope of the present invention. Where no specific techniques or conditions are specified in the examples, the techniques or conditions described in the literature in the art or the product instructions shall be followed. The reagents or instruments used on which no manufacturer is indicated are all commercially available conventional products.

### Synthesis of Compound

### Example 1

In Example 1, Compound A was prepared by using known Compounds 1 and 4. Compounds 1 and 4 were either commercially available or could be synthesized by referring to known routes. For example, Compounds 1 and 4 could be obtained by referring to the content of a Chinese patent with application number 201780004233.0.

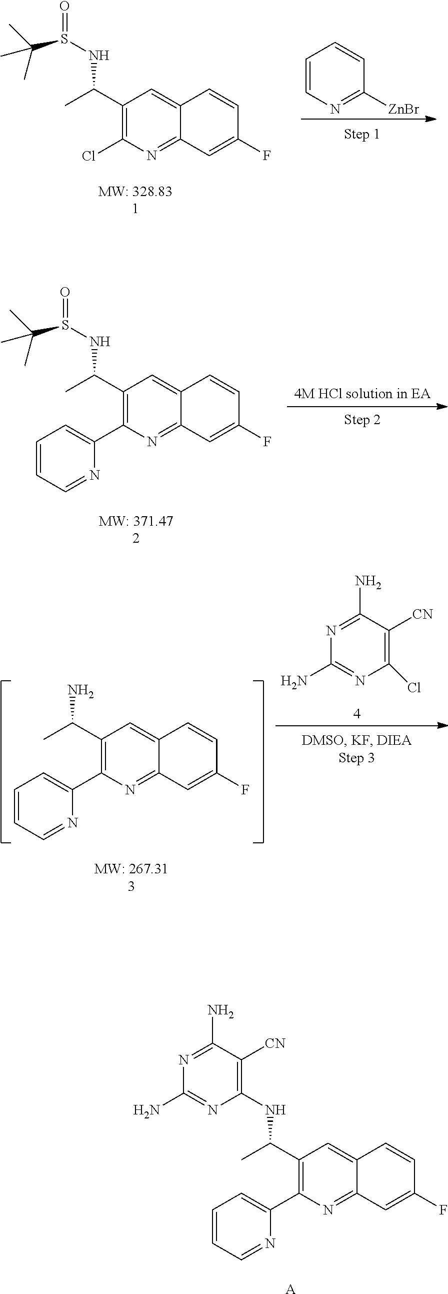

Step 1: i-PrMgCl (13 L) and tetrahydrofuran (THF, 4.0 L) were added to a reaction vessel in an N₂ atmosphere. A THF (4.0 L) solution containing 2-bromopyridine (4.12 kg) was then added at 30° C.±5° C. The mixture was stirred at 30° C.±5° C. for at least 2 h. A solution of ZnBr₂ (7.05 kg) in THF (10 L) was then added, and the reaction system was stirred at 30° C.±10° C. for at least 1 h. Compound 1 (4.3 kg), XPhos (748 g), NaI (198 g) and Pd(AcO)₂ (89 g) were added to the reaction vessel, the obtained mixture was heated to 65° C.±5° C., and the reaction system was stirred at 65° C.±5° C. for at least 24 h. The reaction system was then cooled to 25° C.±5° C. Dichloromethane (DCM, 20 L) was added and stirred for at least 20 min. The resulting mixture was filtered and the filter cake was washed twice with DCM (6.0 L). The organic phase was concentrated and exchanged with DCM to 10 L. An EDTA sodium solution (20 L) and DCM (30 L) were then added, and the reaction system was stirred at 25° C.±5° C. for at least 0.5 h. The resulting mixture was filtered and the filter cake was washed twice with DCM (6.0 L). The filtrate was separated and the organic phase was collected. The organic phase was washed three times with an EDTA sodium solution (20 L). The organic phase was collected, concentrated and exchanged with ethyl acetate (EA) to 4.0-6.0 L. The mixture was cooled to −15° C.±5° C. n-Heptane (40 L) was then added. The mixture was stirred at −15° C.±5° C. for at least 12 h. The solid was filtered and the filter cake was washed twice with n-heptane (6.0 L). If the by-product of dechlorination is >1.0%, the following operations were continued: the filter cake was slurried with EA/n-heptane (4.0 L/40 L). The mixture was stirred at −15° C.±5° C. for at least 8 h. The product was filtered and the filter cake was washed twice with n-heptane (4.0 L). The filter cake was collected and dried at 45° C.±5° C. for at least 16 h. 4.6 kg of a light yellow solid was obtained with a purity of 97.78%. The yield was 95%.

Step 2: EA (22.5 L) and Compound 2 (4.5 kg) were charged into a reaction vessel in an N₂ atmosphere. A solution of 4 M HCl in ethyl acetate (22.5 L) was then added at 20° C.±5° C. The mixture was stirred at 20° C.±5° C. for at least 2 h. The filter cake was filtered and collected. The filter cake and water (45 L) were then mixed. The aqueous phase was washed once with DCM (45 L) and once with methyl tert-butyl ether (MTBE, 45 L). The aqueous phase was adjusted to pH=9 with NH₃·H₂O (about 4.5 L). The aqueous phase was extracted twice with DCM (27 L). 3-Mercaptopropyl ethyl sulfide silica (10%, w/w) was then added. The mixture was stirred at 40° C.±5° C. for at least 2 h. A solid was filtered out and the filter cake was washed twice with DCM (9 L). The organic phase was collected and concentrated to obtain an oil. The residue was directly used in the next step without purification. The yield was 93%.

Step 3: DMSO (10 L), Compound 3 (2.76 kg), Compound 4 (1.85 kg), KF (0.61 kg) and N,N-diisopropylethylamine (DIEA, 2.68 kg) were added to a reaction vessel in an N2 atmosphere. The mixture was heated to 100° C.±5° C. The reaction system was stirred at 100° C.±5° C. for at least 24 h. The mixture was then cooled to 25° C.±5° C. and added to water (83 L). The mixture was stirred for at least 0.5 h and filtered. The solid was collected and dissolved in DCM (33 L). 1.2 N HCl (40 L) was then added and stirred for at least 0.5 h. After separation, the aqueous phase was collected and washed three times with DCM (33 L). The aqueous phase was added to an Na₂CO₃ aqueous solution (1.2 N, 33 L), the mixture was stirred for at least 30 min, a solid was filtered out, and the filter cake was washed twice with water (7 L). The filter cake was collected and dried at 45° C.±5° C. for at least 16 h to obtain Compound A with a yield of 83%. Mass spectrometry (ESI) m/e: 401 (M+1). ¹H NMR (300 MHz, DMSO-d6) ppm 8.72 (s, 1H), 8.56 (m, 1H), 7.54-8.12 (m, 6H), 6.50 (s, 2H), 6.08 (s, br, 2H), 5.65-5.75 (m, 1H), 1.35 (d, J=6.9 Hz, 3H).

### Example 2

In Example 2, Compound A prepared in Example 1 was subjected to formation screening in a 96-well plate salt with 12 acids, and the solid samples prepared in the salt formation screening were analyzed and confirmed by means of nuclear magnetic resonance (1H NMR) and X-ray powder diffraction (XRPD).

The instrument used for ¹H NMR analysis was Bruker Advance 300 equipped with a B-ACS 120 automatic sampling system.

The X-ray diffraction analyzer used for XRPD involved in this example and elsewhere in the description was Bruker D8 advance, which was equipped with a LynxEye detector. The 2θ scanning angle for the sample was from 3° to 40°, and the scanning step was 0.02°. During sample testing, the light tube voltage and current were 40 kV and 40 mA, respectively.

An appropriate amount of Compound A was dissolved in methanol to prepare a drug solution with a concentration of 30 mg/mL.

The acids used in the experiment were as shown in Table 1 below. Certain amounts of the acids were dissolved and diluted with methanol to separately prepare acid solutions with a concentration of 0.1 M.

TABLE 1
Acids for experiment

| | | | |
|---|---|---|---|
| Hydrochloric | Phosphoric acid | P-toluenesulfonic | Methane- |
| acid (HCl) | (H₃PO₄) | acid (p-TsOH) | sulfonic |
| | | | acid |
| Hydrobromic | Maleic acid | Fumaric acid | Citric |
| acid (HBr) | | | acid |
| Sulfuric acid | L-tartaric acid | Benzoic acid | Succinic |
| (H₂SO₄) | | | acid |

The solvents used were as shown in Table 2.

TABLE 2
Solvents for experiment

| | | |
|---|---|---|
| Methanol (MeOH) | Acetonitrile (ACN) | Acetone |
| Isopropanol (IPA) | Ethanol (EtOH) | Water (H₂O) |
| Tetrahydrofuran | Methyl tert-butyl ether | Ethyl acetate |
| (THF) | (MTBE) | (EA) |

The drug solution prepared above was distributed in a 96-well plate, and the acid was then added. Each well contained 100 μL of the drug solution and one acid solution prepared above, and all the other acids were 1.05 equivalents except sulfuric acid which was 0.55 equivalents.

After the liquid in the 96-well plate was evaporated, 200 μL of the solvent required for screening was added to each well. Subsequently, the 96-well plate was sealed with a perforated sealing film and placed in a fume hood at room temperature. After the solvent slowly evaporated, solid samples with good quality were selected and characterized by XRPD and ¹H NMR to determine whether a salt was formed and whether the salt was crystal.

The distribution methods for the acid solutions and solvents were as shown in Table 3. After the liquids in the 96-well plate were evaporated, the state of the samples was as shown in Table 3. Some samples were selected for ¹H NMR and XRPD characterization.

TABLE 3
State of samples in 96-well plate

| | | A | B | C | D | E | F | G | H |
|---|---|---|---|---|---|---|---|---|---|
| | | EtOH | IPA | THF | ACN | MTBE | Acetone | Water | EA |
| 1 | Hydrobromic acid | | | A | A | A | | | A |
| 2 | Hydrochloric acid | | | A | A | A | | A | A |
| 3 | Sulfuric acid | A | A | A | A | A | | A | A |
| 4 | P-toluenesulfonic acid | C | C | *C | C | *C | C | *C | C |
| 5 | Methanesulfonic acid | | | A | | A | | | A |
| 6 | Maleic acid | A | A | A | A | A | | A | A |
| 7 | Phosphoric acid | A | A | A | A | A | | A | A |
| 8 | L-tartaric acid | A | A | A | A | A | | A | A |
| 9 | Fumaric acid | A | A | A | A | A | | A | *A |
| 10 | Citric acid | A | A | | A | A | | A | A |
| 11 | Benzoic acid | | | | | *C | | A | |
| 12 | Succinic acid | | | | | *C | | A | |

Remarks: in Table 3,
A = amorphous;
C = crystal;
and the rest were glassy;
*was XRPD measurement results.

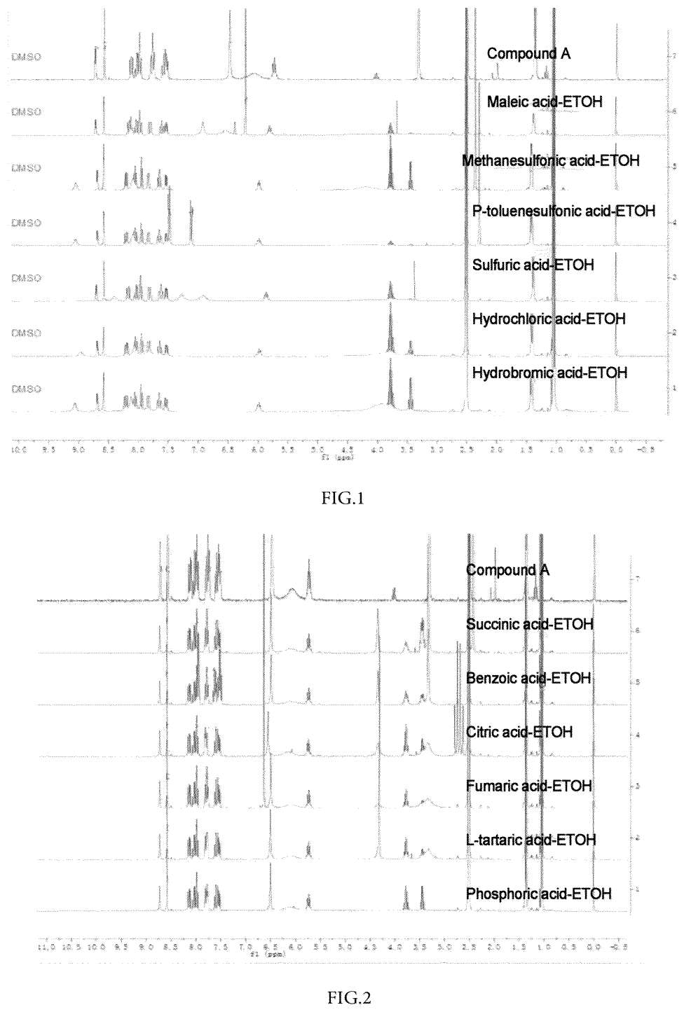

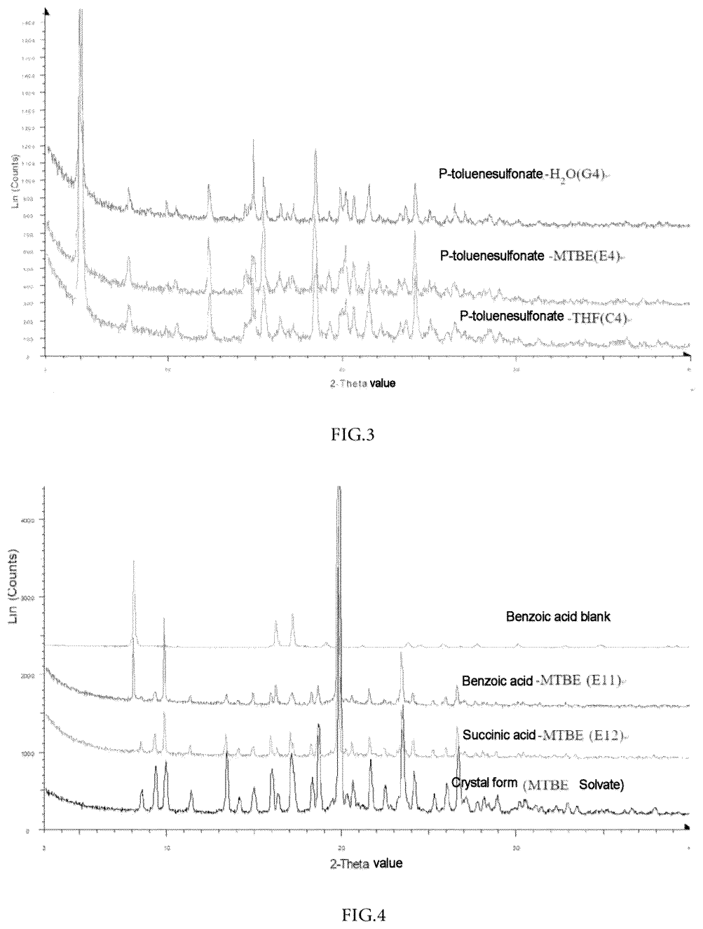

The experimental results showed that Compound A could form salts with hydrochloric acid, hydrobromic acid, sulfuric acid, p-toluenesulfonic acid, methanesulfonic acid and maleic acid. Some of the analysis results were as shown in FIGS. 1-4. In conjunction with the results shown in FIGS. 1 and 2, the reactions of Compound A with hydrochloric acid, hydrobromic acid, sulfuric acid, p-toluenesulfonic acid, methanesulfonic acid and maleic acid all exhibited corresponding chemical shifts in ¹H NMR. In addition, a crystal salt, i.e., p-toluenesulfonate, was obtained in the 96-well plate, and p-toluenesulfonate samples obtained in different solvents were essentially the same in terms of XRPD and were all in crystal form I, as shown in FIG. 3. In addition, although crystal samples were also obtained in wells E11 (benzoic acid-MTBE) and E12 (succinic acid-MTBE), they were judged to be MTBE solvates of Compound A. For example, the detection results of the XRPD patterns thereof were as shown in FIG. 4.

### Example 3

In Example 3, based on the 96-well plate salt formation screening results, various salts were prepared. In the experiment, a certain amount of solvent was added to an appropriate amount of free alkali, and an acid was then added at room temperature or under heating conditions for a salt formation experiment.

### 1. P-toluenesulfonate

As shown in Table 4, p-toluenesulfonates were prepared in different solvents at room temperature or 40° C., and the numbers of the obtained p-toluenesulfonate samples were listed in the first column of Table 4. FIG. 5 showed that samples 1, 2, 3 and 4 prepared in different solvents and the 96-well plate-G4 sample prepared in Example 2 above had essentially the same XRPD patterns, named as p-toluenesulfonate crystal form I.

TABLE 4
Preparation of p-toluenesulfonates

| No. | Acid | Phenomenon |
|---|---|---|
| Sample 1 | P-toluenesulfonic | At room temperature, about 25 mg of Compound A and 1.05 |
| | acid solid | equivalent of p-toluenesulfonic acid solid were added to 10 V of |
| | | acetone and dissolved clear, whereupon a precipitate precipitated |
| | | out rapidly. After stirring for 1 h, filtration was carried out, and the |
| | | obtained sample was dried at 50° C. overnight. |
| Sample 2 | P-toluenesulfonic | At 40° C., about 30 mg of MTBE solvate of Compound A was |
| | acid solid | dissolved in 20 V of acetone, and 1.05 equivalent of p- |
| | | toluenesulfonic acid solid was then added, whereupon a precipitate |
| | | precipitated out immediately; and after stirring at 40° C. for 0.5 h, |
| | | stirring was continued at room temperature for 1 h, filtration was |
| | | then carried out, and the obtained sample was dried at room |
| | | temperature overnight. |
| Sample 3 | 0.75M p- | At room temperature, about 30 mg of MTBE solvate of Compound |
| | toluenesulfonic | A was dissolved in 7 V of acetone, 90 μL (1.05 equivalent) of an |
| | acid aqueous | acid solution was then added, and after stirring at room temperature |
| | solution | for 1 h or longer, a precipitate precipitated out; and after continued |
| | | stirring 3 h, filtration was carried out, and the obtained sample was |
| | | dried at room temperature overnight. |
| Sample 4 | 1.25 M p- | At 40° C., about 25 mg of MTBE solvate of Compound A was |
| | toluenesulfonic | dissolved in 20 V of ethyl acetate, and 46.5 μL (1.05 equivalent) of |
| | acid solution | 1.25M p-toluenesulfonic acid solution in acetone was then added, |
| | in acetone | whereupon a precipitate precipitated out immediately; and after |
| | | continued stirring at 40° C. for 20 min, filtration was carried out, and |
| | | the obtained sample was dried at room temperature overnight. |

Then, Sample 3 was taken as an example for characterization by thermogravimetric analysis (TGA), differential scanning calorimetry (DSC), ¹H NMR and dynamic water desorption analysis (DVS).

The instrument model used for TGA analysis was TA TGA Q500 or Discovery TGA 55 (TA Instruments, US). The sample was placed in a balanced open aluminum sample tray, and the mass was automatically weighed in a TGA heating furnace. The sample was heated to the final temperature at 10° C./min.

The instrument model used for DSC analysis was TA DSC Q200 or Discovery DSC 250 (TA Instruments, US). The sample was precisely weighed and then placed in a perforated DSC sample tray, and the accurate mass of the sample was recorded. The sample was heated to the final temperature at 10° C./min.

The instrument model used for DVS analysis was IGA Sorp (Hidentity Isochema). The sample was measured in a gradient mode, the humidity range for the test was from 0% to 90%, and the humidity increment of each gradient was 10%. The time to keep at each humidity gradient was from 30 min to 2 h.

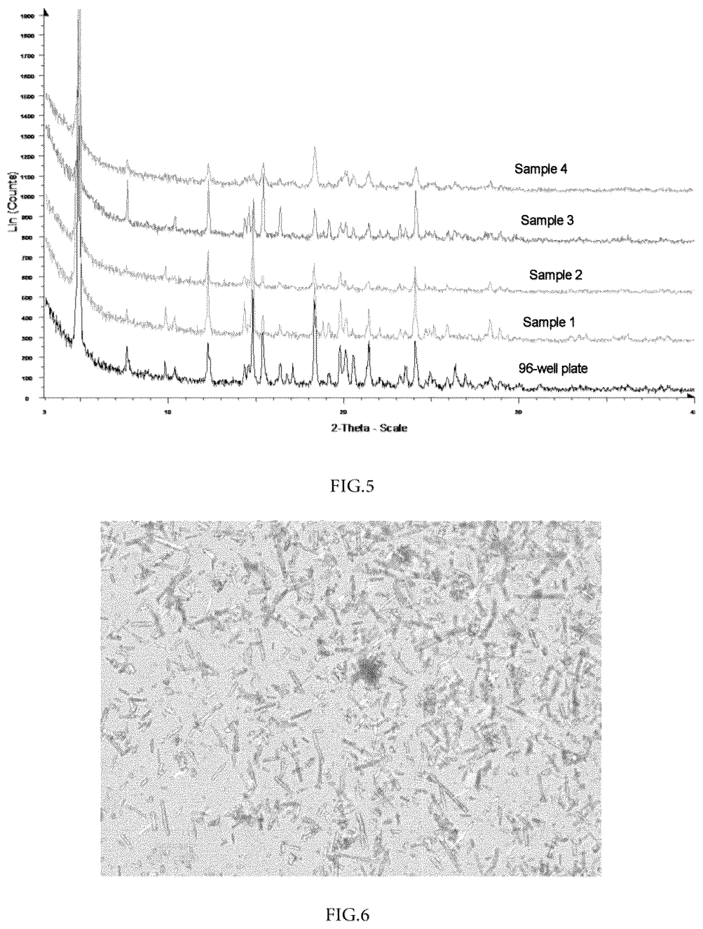

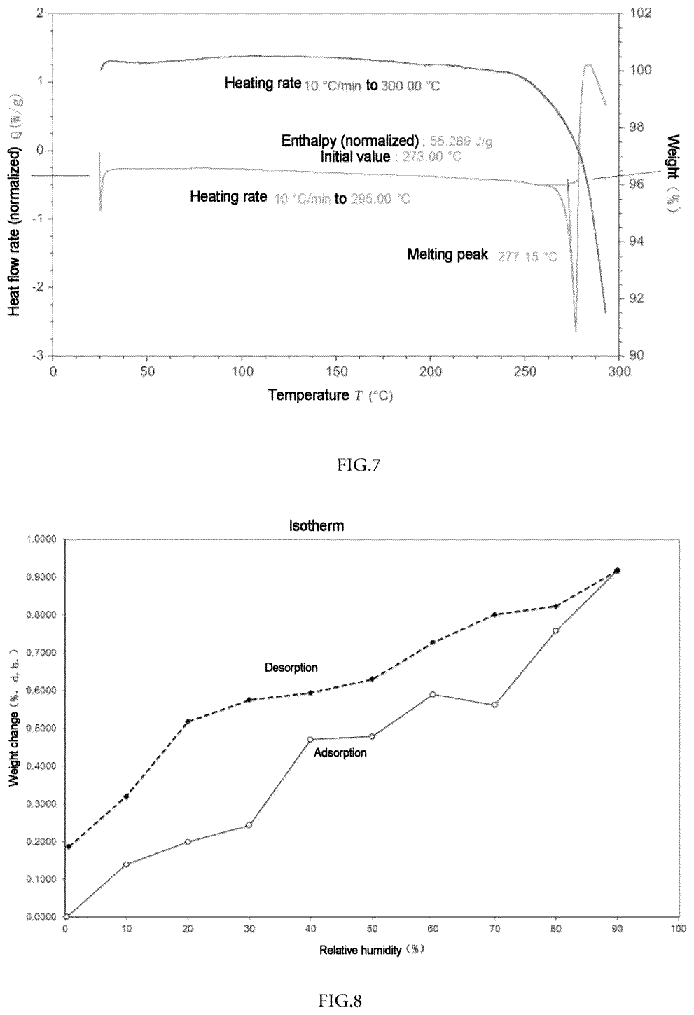

The analysis results were as shown in FIGS. 6-9. It was determined by NMR and TGA that the sample had essentially no weight loss and no solvent residue before decomposition. The DSC pattern in FIG. 7 showed an endothermic peak near 277° C., which should be a melting peak, and immediate decomposition after melting. As shown in FIG. 8, p-toluenesulfonate crystal form I only absorbed 0.92% of moisture even at 90% RH. Moreover, as shown in FIG. 9, the crystal form of the sample remained unchanged before and after humidity change.

### 2. Hydrobromide, Hydrochloride, Sulfate, Methanesulfonate and Maleate

The five salts were prepared below by means of similar methods: About 20 mg of Compound A was added to 10 V of ethyl acetate and dissolved clear, various acids were added separately, and the mixtures were stirred overnight at 50° C. to obtain hydrobromide, hydrochloride, sulfate, methanesulfonate and maleate of Compound A, respectively. After TGA analysis of the obtained methanesulfonate crystal, the results showed a weight loss of 2.40% at 100-170° C. and two endothermic peaks in DSC, with the peak temperatures being 134.5° C. and 195.6° C., respectively. The thermal analysis pattern of the methanesulfonate showed a weight loss and a corresponding endothermic peak, and ¹H NMR showed that about 2.87% of ethyl acetate remained. The prepared methanesulfonate was a solvate, and this crystal form was defined as methanesulfonate crystal form I.

### Example 4

In Example 4, p-toluenesulfonate of Compound A was prepared, and the solubility and stability of crystal form I prepared from p-toluenesulfonate were compared with those of the compound.

The HPLC method used therein was as shown in Table 5 below:

TABLE 5
HPLC detection conditions

| | |
|---|---|
| Instrument | Agilent 1260 series |
| Chromatographic | X Bridge C18 150*4.6 mm, 3.5 um |
| column | |
| Column temperature | 35° C. |
| Mobile phase | A: 0.1% ammonia aqueous solution |
| | B: acetonitrile |
| Gradient conditions | 0 min: 5% |
| (% B) | 12 min: 100% |
| | 15 min: 100% |
| Flow rate | 1.0 mL/min |
| Sample volume | 5 μL |
| Delay time | 3 min |
| Detection wavelength | 254 nm |
| Diluent | Acetonitrile |

The preparation method for p-toluenesulfonate (crystal form I, No. 5) was as follows:

519 mg of Compound A was added to 3.6 mL (7 V) of acetone and dissolved clear, 1.36 mL (1.05 equivalent, about 2V) of 1.0 M p-toluenesulfonic acid aqueous solution was added thereto. After stirring at room temperature for 2 h, there was still no precipitation. After adding 0.1 mg of a seed crystal (Sample 3 in Table 4) thereto, a solid gradually precipitated out. The suspension was stirred at room temperature for 0.5 h and filtered, and the obtained solid was dried overnight at room temperature to obtain Sample 5. After characterization, Sample 5 was p-toluenesulfonate crystal form I, and the melting point thereof was about 280° C.

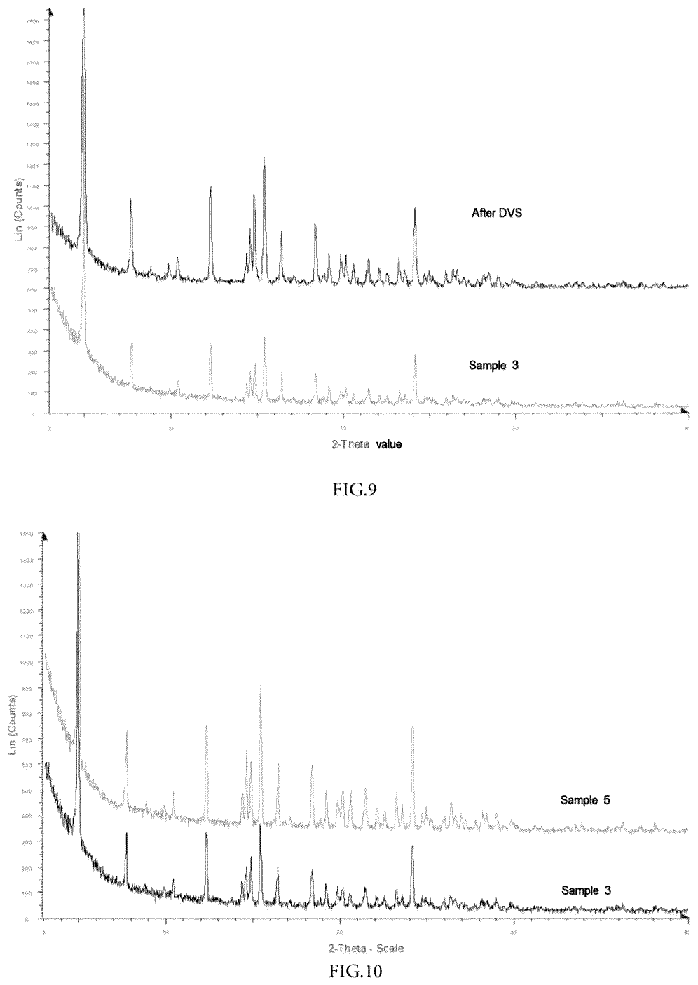

The XRPD pattern results of the prepared Samples 3 and 5 were as shown in FIG. 10.

The preparation method for sample 3 was as follows:

At room temperature, about 30 mg of MTBE solvate of Compound A was dissolved in 7 V of acetone, 90 μL (1.05 equivalent) of a 0.75 M p-toluenesulfonic acid aqueous solution was then added, and after stirring at room temperature for 1 h or longer, a precipitate precipitated out; and after continued stirring 3 h, filtration was carried out, and the obtained sample was dried at room temperature overnight.

The XRPD pattern results of the prepared Samples 3 and 5 were as shown in FIG. 10, and they were both crystal form I.

Sample 3 was taken as an example for dynamic water desorption analysis (DVS) characterization. The instrument model used for DVS analysis was IGA Sorp (Hidentity Isochema). The sample was measured in a gradient mode, the humidity range for the test was from 0% to 90%, and the humidity increment of each gradient was 10%. The time to keep at each humidity gradient was from 30 min to 2 h. The results thereof were as shown in FIG. 11. The test results showed that Sample 3 only absorbed 0.92% of moisture even at 90% RH and exhibited an extremely low hygroscopicity.

The solubility test conditions were as follows:

The solubilities of Compound A and p-toluenesulfonate (Sample 5, crystal form I) in simulated gastrointestinal fluids (SGF, FeSSIF and FaSSIF) at 37° C. were tested.

About 7.5 mg of Compound A and p-toluenesulfonate were separately added to 1.5 mL of three biological vehicles to prepare suspensions. All the suspensions were placed in a shaker at 37° C. and shaken at 200 rpm for 24 h, and about 0.5 mL was sampled separately at 0.5 h, 2 h and 24 h for filtration. The obtained filtrates were subjected to HPLC analysis and pH measurement, and the filter cakes were respectively measured by XRPD.

The results thereof were as follows:

TABLE 6
Solubility results

| | | Solubility (mg/mL) | | | pH- | pH- | pH- |
|---|---|---|---|---|---|---|---|
| Sample | Medium | 30 min | 2 h | 24 h | 0.5 h | 2 h | 24 h |
| Compound A | SGF | >4.92 | >4.91 | >4.83 | 1.19 | 1.16 | 1.18 |
| | (pH 1.21) | | | | | | |
| | FeSSIF | 2.06 | 1.89 | 2.20 | 5.00 | 4.94 | 4.86 |
| | (pH 4.99) | | | | | | |
| | FaSSIF | 0.37 | 0.32 | 0.35 | 6.55 | 6.51 | 6.34 |
| | (pH 6.52) | | | | | | |
| Sample 5 | SGF | >5.44 | >5.40 | >5.35 | 1.05 | 1.10 | 1.11 |
| | (pH 1.21) | | | | | | |
| | FeSSIF | 1.31 | 2.74 | 3.57 | 4.81 | 4.80 | 4.69 |
| | (pH 4.99) | | | | | | |
| | FaSSIF | 0.57 | 0.56 | 0.55 | 6.27 | 6.10 | 5.38 |
| | (pH 6.52) | | | | | | |

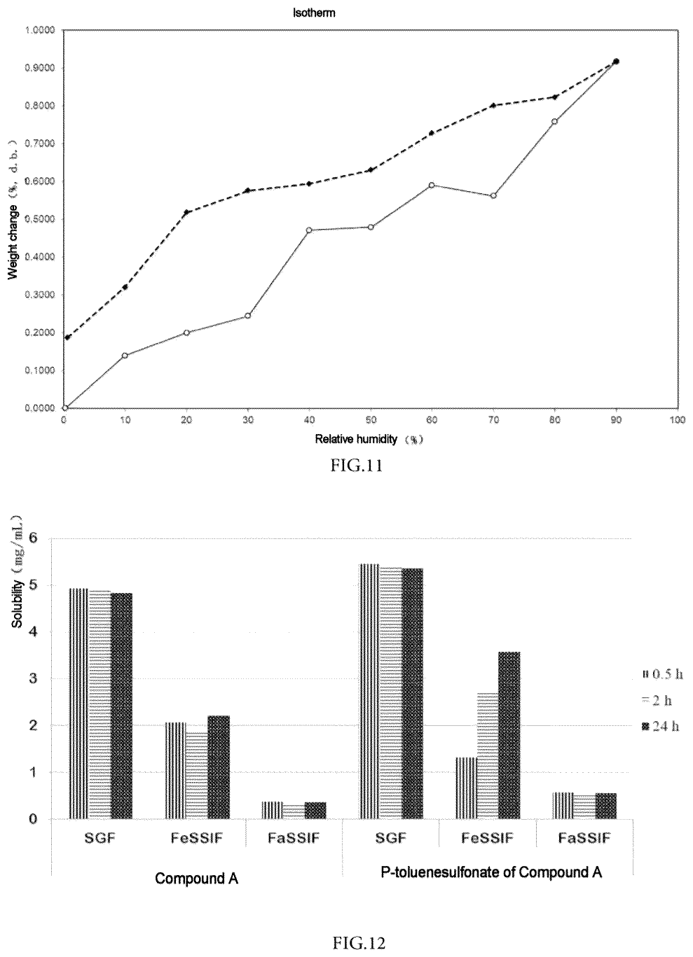

As shown in Table 6 and FIG. 12, the solubilities of Compound A and p-toluenesulfonate of Compound A (Sample 5, crystal form I) were pH-dependent and increased with the decrease of pH. Compound A and p-toluenesulfonate of Compound A had higher solubilities in SGF and FeSSIF, and the solubility of p-toluenesulfonate of Compound A in the three media was generally higher than that of Compound A.

The stability test conditions were as follows:

Appropriate amounts of Compound A and p-toluenesulfonate of Compound A (Sample 5, crystal form I) were respectively placed in 40° C./75% RH and 60° C. environments for one week to determine solid stability. At days 0 and 7, HPLC purity analysis and solid XRPD determination were carried out, and some of the results were as shown in Table 7.

TABLE 7
Solid stability results

| | Purity (peak area %) | | | | | |
|---|---|---|---|---|---|---|
| | | | | Average - | | |
| | | Average - | 40° C./75% | 40° C./75% | | Average - |
| Sample | 0 d | 0 d | RH - 7 d | RH - 7 d | 60° C. - 7 d | 60° C. - 7 d |
| Compound A | 98.63 | 98.59 | 98.52 | 98.49 | 98.37 | 98.43 |
| | 98.55 | | 98.45 | | 98.50 | |
| Sample 5 | 99.46 | 99.48 | 99.68 | 99.69 | 99.56 | 99.65 |
| | 99.49 | | 99.69 | | 99.74 | |

After being placed under the conditions of 40° C./75% RH and 60°C for 7 days, the p-toluenesulfonate of Compound A exhibited excellent physical and chemical stability and had no change in purity and crystal form. However, the chemical purity of the compound itself decreased by about 0.16% after being placed at 60° C. for 7 days.

The salt of the compound shown in Formula (I) provided by the present invention easily crystallized, had acceptable physical and chemical properties, and had higher chemical stability than the compound itself.

### Example 5

In this example, the instrument model used for TGA analysis was TA TGA Q500 or Discovery TGA 55 (TA Instruments, US). The sample was placed in a balanced open aluminum sample tray, and the mass was automatically weighed in a TGA heating furnace. The sample was heated to the final temperature at 10° C./min.

The instrument model used for DSC analysis was TA DSC Q200 or Discovery DSC 250 (TA Instruments, US). The sample was precisely weighed and then placed in a perforated DSC sample tray, and the accurate mass of the sample was recorded. The sample was heated to the final temperature at 10° C./min.

In Example 5, referring to the salt formation screening experiment, p-toluenesulfonate of Compound A was prepared according to the following method:

At room temperature, about 1.40 g of MTBE solvate of Compound A was dissolved in 9.8 mL (7 V) of acetone, and 3.05 mL (1.05 equivalent, about 2 V) of a 1 M p-toluenesulfonic acid/(acetone/water=3/1) solution was then added, whereupon a precipitate precipitated out immediately; and stirring continued for 0.5 h, a solid was filtered out, and the obtained sample was dried overnight at room temperature. 1.24 g of p-toluenesulfonic acid solid (i.e., Sample 7) was obtained. At room temperature, about 10 V of water was added to about 1.10 g of p-toluenesulfonate (Sample 7), and after stirring at room temperature for 2 h, a solid was filtered out and dried at 50° C. overnight. About 1 g of p-toluenesulfonate (i.e., Sample 8) was obtained.

According to the XRPD results, the crystal form of Sample 8 was characterized as crystal form I, as shown in FIG. 13. The TGA-DSC pattern results of Sample 8 were as shown in FIG. 14. As determined by TGA, the sample had essentially no weight loss before decomposition, and the ¹H NMR results showed no residual organic solvent (as shown in FIG. 15). The DSC pattern showed an endothermic peak at 283° C., which should be a melting peak, and immediate decomposition after melting.

TABLE 8
XRPD characterization results

| | Relative |
|---|---|
| 2-theta ° | intensity % |
| 4.876 | 100 |
| 7.631 | 9.3 |
| 8.752 | 2.6 |
| 9.839 | 4.1 |
| 10.33 | 3.9 |
| 12.241 | 13.6 |
| 14.318 | 5.1 |
| 14.542 | 5.6 |
| 14.819 | 11.8 |
| 15.364 | 10.3 |
| 16.346 | 5.1 |
| 17.056 | 1.5 |
| 17.634 | 1.4 |
| 18.32 | 4.9 |
| 18.788 | 1.8 |
| 19.128 | 3.2 |
| 19.816 | 3.9 |
| 20.086 | 2.6 |
| 20.547 | 2 |
| 21.411 | 3 |
| 22.061 | 1.8 |
| 22.488 | 1.7 |
| 23.206 | 2.1 |
| 23.499 | 1.7 |
| 24.089 | 6 |
| 24.702 | 1.8 |
| 25.199 | 1.3 |
| 25.932 | 1.3 |
| 26.338 | 1.6 |
| 27.752 | 1 |
| 28.128 | 1.3 |
| 28.389 | 1.5 |
| 28.945 | 1.7 |
| 29.805 | 1 |
| 33.035 | 0.8 |
| 33.469 | 0.8 |
| 33.826 | 0.8 |
| 35.855 | 0.7 |
| 36.24 | 0.8 |
| 38.043 | 0.7 |
| 38.553 | 0.8 |

Then, the prepared Sample 8 was used as a starting material for crystal form screening. In the experiment, Sample 8 was subjected to crystal screening by means of a variety of methods such as suspension beating, anti-solvent precipitation, cooling crystallization and volatile crystallization. It was found from the experiment that no new crystal form appeared except crystal form I during the screening process.

### 1. Suspension Beating Experiment

At room temperature or 50° C., the suspension beating experiment was tried in various solvents.

### (1) Suspension Beating Experiment in Single Solvent

At room temperature, about 25 mg of p-toluenesulfonate crystal form I was weighed, suspended and stirred in individually 10 solvents (20 V) for 2 days, and the samples with 10 unchanged crystal form were heated to 50° C. to continue suspension under stirring for 1 day. The obtained samples were measured by XRPD, and the results were as shown in Table 9.

TABLE 9
Results of suspension beating experiment in single solvent

| ID | Solvent | RT - 2 d | 50° C. - 1 d |
|---|---|---|---|
| 1 | Toluene | Crystal form I | Crystal form I |
| 2 | n-Heptane | Crystal form I | Crystal form I |
| 3 | Cyclohexane | Crystal form I | Crystal form I |
| 4 | Methyl tert-butyl | Crystal form I | Crystal form I |
| | ether | | |
| 5 | Isopropyl acetate | Crystal form I | Crystal form I |
| 6 | Isopropanol | Crystal form I | Crystal form I |
| 7 | Ethyl acetate | Crystal form I | Crystal form I |
| 8 | Ethanol | Crystal form I | Crystal form I |
| 9 | Acetonitrile | Crystal form I | Crystal form I |
| 10 | Butanone | Crystal form I | Crystal form I |

The experimental results showed that Sample 8 was subjected to the suspension beating experiment in a single solvent, and the crystal forms of the obtained samples were all p-toluenesulfonate crystal form I.

### (2) Suspension Beating Experiment in Mixed Solvents

About 15 mg of p-toluenesulfonate crystal form I (Sample 8) sample was added to 0.3 or 0.5 mL of 16 mixed solvents respectively to prepare suspensions. The obtained suspensions were respectively stirred at room temperature for 4 days or shaken at 50° C. for 1 day, and the obtained solid samples were measured by XRPD.

TABLE 10
Results of suspension beating experiment in mixed solvents

| | | Volume | Temperature/ | |
|---|---|---|---|---|
| No. | Solvent | (μL) | Time | Results |
| 1 | Methanol/isopropyl | 500 | RT/4 d | Crystal |
| | acetate (1/2) | | | form I |
| 2 | Methanol/isopropanol | 300 | RT/4 d | Crystal |
| | (1/10) | | | form I |
| 3 | Methanol/ethyl acetate | 500 | RT/4 d | Crystal |
| | (1/2) | | | form I |
| 4 | Tetrahydrofuran/iso- | 300 | RT/4 d | Crystal |
| | propanol (1/2) | | | form I |
| 5 | Tetrahydrofuran/water | 500 | RT/4 d | Solution |
| | (1/2) | | | |
| 6 | Isopropanol/water (1/4) | 500 | RT/4 d | Crystal |
| | | | | form I |
| 7 | Isopropanol/water (1/1) | 500 | RT/4 d | Crystal |
| | | | | form I |
| 8 | Isopropanol/water (9/1) | 300 | 50° C./1 d | Crystal |
| | | | | form I |
| 9 | Isopropanol/water (1/2) | 300 | 50° C./1 d | Crystal |
| | | | | form I |
| 10 | Isopropanol/water (1/9) | 300 | 50° C./1 d | Crystal |
| | | | | form I |
| 11 | Water | 300 | 50° C./1 d | Crystal |
| | | | | form I |

The experimental results were as shown in Table 10. After the suspension beating experiment in mixed solvents, the obtained solid samples were all crystal form I, and no new crystal form was found.

### 2. Cooling Crystallization

In the experiment, taking ethanol, butanone and acetone as examples, p-toluenesulfonate crystal form I was subjected to a crystallization experiment. The specific experiments and results were as shown in Table 11. At 60° C., 10 mg of Sample 8 was taken and dissolved in different solvents, and a clear solution was obtained only in ethanol. The solution/suspension was filtered, and the filtrate was slowly cooled to room temperature.

TABLE 11
Cooling crystallization experiment

| | | Solvent volume | |
|---|---|---|---|
| No. | Solvent | (mL) | Results |
| 1 | Ethanol | 1 | Crystal form I |
| 2 | Butanone | 2 | Crystal form I |
| 3 | Tetrahydrofuran | 0.7 | Solution |

The experimental results showed solid samples obtained in three solvents (ethanol, butanone and acetone), all of which were p-toluenesulfonate crystal form I.

### 3. Anti-Solvent Precipitation Method

In the experiment, the solubility of p-toluenesulfonate was measured to be >24 mg/ml in methanol and >11.6 mg/mL in tetrahydrofuran. Therefore, these two solvents were taken, for example, as good solvents and subjected to anti-solvent precipitation experiments.

At room temperature, about 10 mg of Sample 8 was dissolved in 0.4 mL of methanol or 0.8 mL of tetrahydrofuran, an anti-solvent was gradually added while stirring, and the precipitated solid sample was characterized by XRPD.

TABLE 12
Anti-solvent precipitation experiment

| | | | | Solid | |
|---|---|---|---|---|---|
| | | | | precipitation | |
| No. | Good solvent | Poor solvent | | time | Results |
| 006 S2 | Methanol | Methyl tert- | 3000 μL | About 20 min | Crystal |
| | (400 μL) | butyl ether | | | form I |
| 006 S3 | | Isopropyl | 7000 μL | >2 d and <5 d | Crystal |
| | | acetate | | | form I |
| 006 S4 | | Isopropanol | 7000 μL | >2 d and <5 d | Crystal |
| | | | | | form I |
| 006 S7 | Tetrahydro- | Toluene | 3000 μL | About 20 min | Crystal |
| | furan | | | | form I |
| 006 S8 | (800 μL) | Methyl tert- | 3000 μL | About 20 min | Crystal |
| | | butyl ether | | | form I |
| 006 S9 | | Isopropyl | 3000 μL | About 20 min | Crystal |
| | | acetate | | | form I |
| 006 S12 | | n-Heptane | 1200 μL | Instant | Crystal |
| | | | | | form I |

The results were as shown in Table 12. The obtained solid samples were all crystal form I, and no new crystal form was found.

In addition, the stability of p-toluenesulfonate crystal form I (Sample 8) under grinding and high humidity conditions was investigated.

A certain amount of p-toluenesulfonate crystal form I was ground in a mortar for 2 min and then measured by XRPD. After grinding, the sample was still p-toluenesulfonate crystal form I.

The p-toluenesulfonate crystal form I sample was essentially non-hygroscopic under high humidity conditions, and the crystal form exhibited a high stability. For example, the p-toluenesulfonate crystal form I sample was placed at RT/92.5% RH for 11 days and then measured by XRPD. As shown in the XRPD pattern shown in FIG. 16, it was found from the results that the crystal form of the sample did not change, and it was still crystal form I. It could be seen therefrom that p-toluenesulfonate crystal form I had a certain stability under high humidity conditions.

In summary, various methods were used to screen the samples of p-toluenesulfonate of Compound A for various crystal forms. The experimental results showed that no other new crystal forms were found in the samples obtained by means of various conditions or solvents except crystal form I. Moreover, the crystal form showed no crystal form change after 11 days at RT/92.5% RH.

P-toluenesulfonate crystal form I had a higher crystallinity, a higher melting point and an extremely low hygroscopicity and was easily obtained by means of reaction and crystallization in acetone/water. To sum up, p-toluenesulfonate crystal form I had relevant properties suitable for subsequent development.

### Example 6

In Example 6, p-toluenesulfonate of Compound A was prepared. The experimental steps were as follows:

(1) A p-toluenesulfonic acid aqueous solution (10 L) was added to a reaction vessel at 27° C.±5° C., and a solution of Compound A (3.37 kg) in ethyl acetate (27 L) was then added. The mixture was stirred at 27 C.±5° C. for at least 12 h. A solid was filtered and the filter cake was collected. The filtrate was separated and the organic phase was collected. The organic phase was adjusted to pH 9-10 with a saturated Na₂CO₃ aqueous solution, and the organic phase was separated and collected. The aqueous phase was extracted once with ethyl acetate (17 L). The organic phases were combined and concentrated to obtain Compound A.

(2) The p-toluenesulfonic acid aqueous solution (10 L) was added to a reaction vessel at 27° C.±5° C., and the solution of Compound A in ethyl acetate (27 L) finally prepared above in Step (1) was then added. The mixture was stirred at 27° C.±5° C. for at least 12 h. The filter cake was filtered and collected. The filter cake obtained in (1) was combined with the filter cake collected here and dried at 45° C.±5° C. for at least 6 h until LOD <5%. The obtained solid was dissolved with purified water (14 L), ethyl acetate (20 L) and acetone (20 L). The solution was then concentrated to 30-36 L. The solution was exchanged three times with ethyl acetate (34 L) for acetone. The reaction system was concentrated to 30-33 L. A small amount of a seed crystal of p-toluenesulfonate of Compound A (Sample 5, 2%, w/w) was added. The reaction system was cooled to 25° C.±5° C. and stirred for at least 12 h. The reaction product was then centrifuged and the filter cake was washed twice with water (6.7 L). The filter cake was collected and dried at 40° C.±5° C. for at least 16 h to obtain Sample 6, i.e., p-toluenesulfonate of Compound A. The yield was 64%.

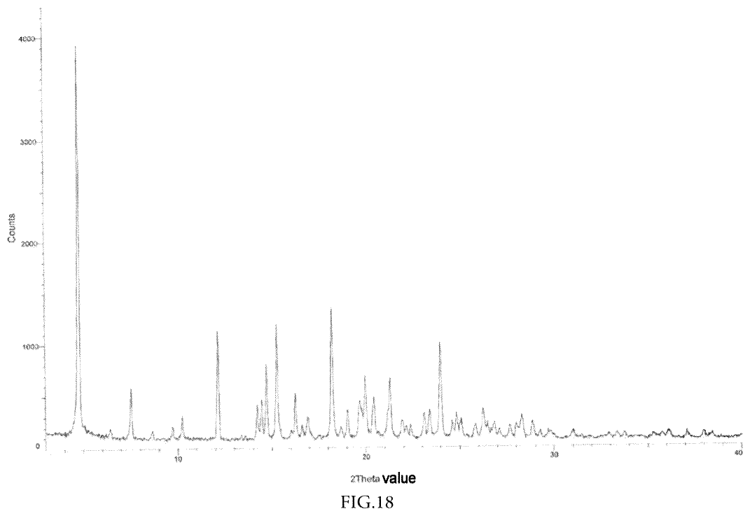

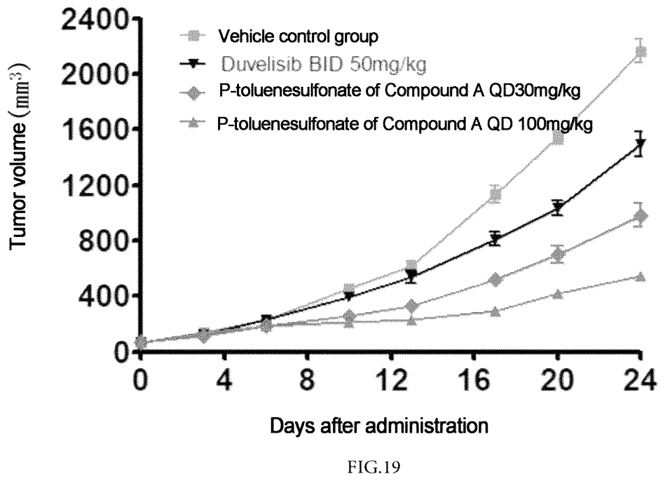

The ¹H NMR and XRPD characterization results of Sample 6 were as shown in FIGS. 17 and 18, respectively.

### Example 7

In Example 7, the inhibitory effects of Sample 6 prepared in Example 6 above on kinases PI3Kα, PI3Kβ, PI3Kγ and PI3Kδ were tested.

The kinases used in the experiment were respectively purchased from:
PI3Kα (p110α/p85α), purchased from Invitrogen, under Cat. No. PV4788;
PI3Kβ (p110β), purchased from eurofins, under Cat. No. 14-603M;
PI3Kδ (p110δ/p85a), purchased from Invitrogen, under Cat. No. PV6452; and
PI3Kγ (p110γ), purchased from Invitrogen, under Cat. No. PR₈₆₄₁C.
Firstly, a 1× kinase buffer solution was prepared, which comprised:
   50 mM HEPES, pH 7.5
   3 mM MgCl₂
   1 mM EGTA
   100 mM NaCl
   0.03% CHAPS
   2 mM DTT

### Preparation of Sample 6 Solution:

On kinases PI3Kα, PI3Kβ and PI3Kγ, the final detection concentration of the compound was 10 μM. 100× concentration was prepared, i.e., 1000 μM. 90 μL of 100% DMSO was added to the second well in row A on a 96-well plate, 10 μL of a 10 mM compound solution was then added, and 3-fold serial dilution was made to obtain a total of 10 concentrations.

On kinase PI3Kδ, the final detection concentration of the compound was 1 μM. 100× concentration was prepared, i.e., 100 μM. 90 μL of 100% DMSO was added to the second well in row B on a 96-well plate, 10 μL of a 1000 μM compound solution was then taken from the second well in row A, and 3-fold serial dilution was made to obtain a total of 10 concentrations.

50 μL of 100% DMSO was transferred to two empty wells as Max well and Min well, respectively.

50 nL of the compound was transferred into a 384-well plate using ECHO550.

### The Reaction Process was as Follows:

Preparation of 2× kinase solution: the kinase was added to a 1× kinase buffer to prepare a 2× enzyme solution.

Addition of enzyme solution to 384-well plate: 2.5 μL of a 2× enzyme solution was added to a 384-well reaction plate, wherein 2.5 μL of a kinase buffer was added to a negative control well, and the plate was incubated for 10 min at room temperature.

Preparation of 2× substrate solution: the kinase was added to a 1× kinase buffer to prepare a 2× substrate solution.

Addition of substrate solution to 384-well plate: 2.5 μL of a 2× substrate solution was added to a 384-well reaction plate.

Kinase reaction: the reaction was carried out at room temperature for 60 min.

Detection of kinase reaction: ADP-Glo reagents were balanced to room temperature, 5 μL of ADP-Glo reagent 1 was transferred to reaction wells of the 384-well plate to stop the reaction, the plate was shaken at 450 rpm for 180 min, 10 μL of ADP-Glo reagent 2 (detection reagent) was then transferred to each reaction well, the plate was shaken at 450 rpm for 1 min and left to stand at room temperature for 30 min. The ADP-Glo reagents were purchased from Promege, under Cat. No. v9102.

Finally, the chemiluminescence values were read from Envision 2104 Multi-label Reader and subjected to curve fitting, and IC50 was calculated. The experimental results were as shown in Table 13.

In addition, taking Sample 6 as an example, the inhibitory effects thereof on PI3Kα, PI3Kβ, PI3Kγ and PI3Kδ in corresponding cells were determined. The activity of PI3Kα was detected by the phosphorylation level of Akt in C2Cl2 cells stimulated by IGF-1, the activity of PI3Kβ was detected by the phosphorylation level of Akt in PC-3 cells stimulated by LPA, the activity of PI3Kγ was detected by the phosphorylation level of Akt in Raw264.7 cells stimulated by c5α, and the activity of PI3Kδ was detected by the phosphorylation level of Akt in Raji cells stimulated by IgM. The phosphorylation level of Akt in cells was determined by means of AlphaLISA technique from PerkinElemer.

The C2C12 cells, PC-3 cells, Raw264.7 cells and Raji cells were all purchased from ATCC. The results were as shown in Table 13.

TABLE 13
Biological activity and selectivity results

| IC50 (nM) | PI3Kδ | PI3Kγ | PI3Kβ | PI3Kα |
|---|---|---|---|---|
| Biochemical | 0.49 | 4.8 | 44 | 117 |
| Cell-based | 0.95 | 29 | 126 | 3523 |

The above results showed that the provided p-toluenesulfonate of Compound A exhibited inhibitory activity on PI3Kδ and also PI3Kγ. The p-toluenesulfonate of Compound A presented selectivity in inhibiting the activity of phosphoinositide 3-kinases.

Taking p-toluenesulfonate of Compound A as an example, it was found from the experimental study that the p-toluenesulfonate of the compound exhibited inhibitory effects on a variety of tumor cells, e.g., on the proliferation of lymphoma cell lines (such as DoHH-2, SU-DHL-4, SU-DHL-4, SU-DHL-6 and WSU-DLCL-2) in vitro, with an absolute IC50 (AbsIC50) value less than 0.1 micromole. Moreover, it exhibited a significant inhibitory effect on the tumor volume growth in various animal models, such as mouse breast cancer 4T1 subcutaneous transplantation tumor model and mouse colorectal cancer CT26. WT cell subcutaneous transplantation tumor model.

### Example 8

In Example 8, the anti-tumor effects of p-toluenesulfonate of Compound A at various dosages by intragastric administration on human-derived lymphoma DoHH-2 cell line subcutaneous transplantation CB17/SCID female immunodeficient mouse model were evaluated.

7-9-week-old CB17/SCID female immunodeficient mice were subcutaneously inoculated with 5*106 DoHH-2 cells to establish a subcutaneous human lymphoma xenograft tumor model. When the average tumor volume in the mice reached 68 mm³, the mice were randomly divided into groups and dosed on the day of grouping. The treatment method for each treatment group and vehicle control group was as follows:

p-toluenesulfonate of Compound A (Sample 6), given 100 mg/kg (p.o. QD) group, once daily, for 25 days (days 0-24);

p-toluenesulfonate of Compound A (Sample 6), given 30 mg/kg (p.o. QD) group, once daily, for 25 days (days 0-24);

positive treatment group (Duvelisib, purchased from Shanghai Lollane biological technology Co., Ltd.), given 50 mg/kg (p.o. BID) twice daily with an interval of 12 h, for 24 days; and

vehicle control group (5% DMSO/40% PEG400/55% water, by volume) (p.o. QD), given once daily, for 25 days (days 0-24).

There were 10 mice in each group, and all the mice were administered starting on the day of grouping (Day 0) and euthanized on Day 24 after grouping (Day 24). The efficacy was evaluated according to the tumor volume at the end point of the experiment.

The abbreviation p.o. stood for oral gavage, QD stood for once daily, and BID stood for twice daily.

The experimental results were as shown in FIG. 19. The average tumor volume in the mice of the vehicle control group was 2163.13 mm³ on Day 24 after administration. At the doses of p-toluenesulfonate of Compound A of 100 mg/kg (QD) and 30 mg/kg (QD), the average tumor volumes were 549.05 mm³ and 984.45 mm³ on Day 24 after administration, which was statistically significantly different as compared with the vehicle control group (P<0.001), and the relative tumor inhibition rates (TGI) (%) were 75% and 54. For the Duvelisib (50 mg/kg BID) group, the average tumor volume was 1496.12 mm³ on Day 24 after administration, which was statistically significantly different as compared with the vehicle control group (P=0.01), and the TGI (%) was 30%. The results showed that p-toluenesulfonate of Compound A exhibited a significant inhibitory effect on tumor growth in the subcutaneous human lymphoma DoHH-2 xenograft CB17/SCID female immunodeficient mouse model at both of the doses of 100 mg/kg and 30 mg/kg, and the tumor growth inhibitory effect was dose-dependent.

The relative tumor inhibition rate (TGI) was calculated by the following formula:

TGI %=(1−T/C)*100%

T and C were respectively the average tumor volumes in the experimental group and the vehicle control group at a specific time point.

In the explanation of the description, reference to the terms “one example”, “some embodiments”, “one embodiment”, etc., means that specific features, structures, materials or features described in connection with this example or instance are included in at least one example or instance of the present invention. In the description, the schematic expressions of the above-mentioned terms are not necessarily aimed at the same example or instance. Moreover, the specific features, structures, materials or features described can be combined in any one or more examples or instances in a suitable manner. In addition, without conflict, those skilled in the art can incorporate and combine different examples or instances and the features of the different examples or instances described in the description without contradicting each other.

Although the examples of the present invention have been shown and described above, it can be understood that the above examples are exemplary and cannot be understood as limitations to the present invention, and those of ordinary skill in the art can make changes, modifications, substitutions and variations to the above examples within the scope of the present invention.

1. A salt, characterized in that the salt is an organic acid salt or inorganic acid salt of a compound shown in Formula (I), wherein
the organic acid comprises at least one selected from toluenesulfonic acid, benzenesulfonic acid, methanesulfonic acid, ethanesulfonic acid and maleic acid;
the inorganic acid comprises at least one selected from hydrochloric acid, hydrobromic acid and sulfuric acid; and
the compound shown in Formula (I) is 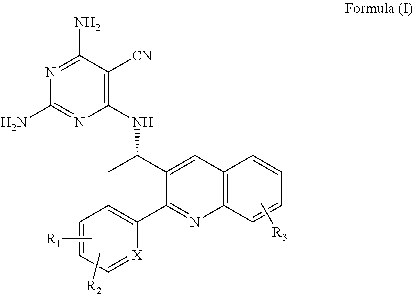
in which X is selected from N or CH;
each of R₁ and R₂ is independently selected from H, F, and SO₂Me, and R₃ is selected from F and Cl.

2. The salt according to claim 1, characterized in that the organic acid comprises at least one selected from toluenesulfonic acid and methanesulfonic acid;
preferably, the organic acid is p-toluenesulfonic acid, m-toluenesulfonic acid or o-toluenesulfonic acid; and
preferably, X is N, each of R₁ and R₂ is H, and R₃ is 7-F.

3. A p-toluenesulfonate of Compound A, characterized in that the Compound A is:

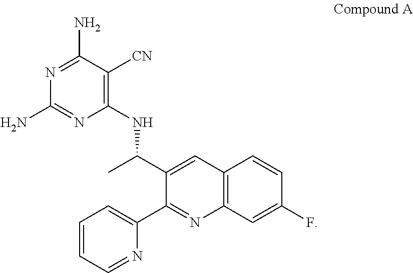 [SMILES: C[C@H](Nc1nc(N)nc(N)c1C#N)c1cc2ccc(F)cc2nc1-c1ccccn1]

4. A pharmaceutical composition, characterized in that the pharmaceutical composition comprises the salt according to either of claims 1 and 2 or the p-toluenesulfonate of Compound A according to claim 3, and a pharmaceutically acceptable carrier.

5. A method for preparing the salt according to either of claims 1 and 2, characterized in that the method comprises:
reacting the compound shown in Formula (I) with an organic acid or inorganic acid to form the salt, wherein
preferably, the reaction is carried out at 20-50 degrees Celsius, and
further preferably, the reaction is carried out at 25-40 degrees Celsius.

6. The method according to claim 5, characterized in that the method further comprises:
preparing a reaction product of a compound shown in Formula (I) and an organic acid or inorganic acid in an organic solvent; and
recovering a solid from the reaction product by filtration, wherein
preferably, the organic solvent comprises at least one selected from methanol, isopropanol, tetrahydrofuran, 2-methyltetrahydrofuran, acetonitrile, ethanol, methyl tert-butyl ether, acetone and ethyl acetate.

7. A method for preparing a compound shown in Formula (I), characterized in that the method comprises:
reacting a compound shown in Formula (II) with a compound shown in Formula (III) to form a compound shown in Formula (IV);
reacting the compound shown in Formula (IV) with an acid to form a compound shown in Formula (V); and
reacting the compound shown in Formula (V) with 2,4-diamino-6-chloropyrimidine-5-carbonitrile to form the compound shown in Formula (I); 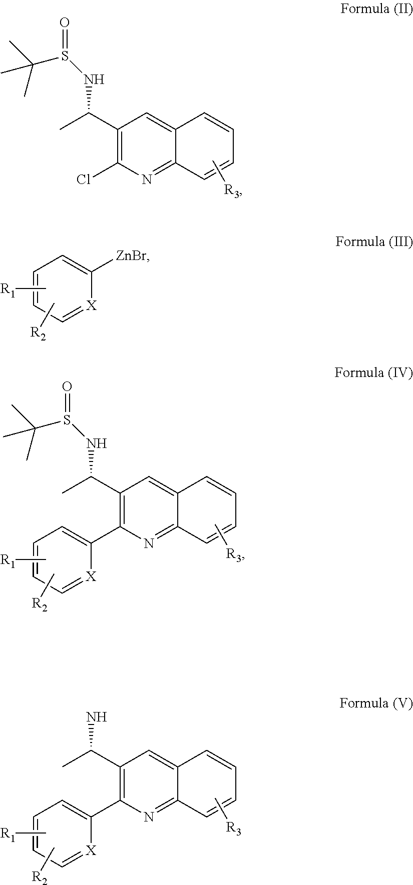
wherein the group R₃ in each of the compounds shown in Formulas (II), (IV) and (V) is the same as the group R₃ in the compound shown in Formula (I);
the groups R₁ and R₂ in the compounds shown in Formulas (III), (IV) and (V) are respectively the same as the groups R₁ and R₂ in the compound shown in Formula (I); and
X in the compounds shown in Formulas (III), (IV) and (V) is selected from N or CH.

8. Use of the salt according to either of claims 1 and 2 or the p-toluenesulfonate of Compound A according to claim 3 or the pharmaceutical composition according to claim 4 in the preparation of a medicament for preventing or treating a disease related to phosphoinositide 3-kinase.

9. A method for selectively inhibiting the growth or proliferation of cells containing phosphoinositide 3-kinase in vitro, characterized in that the method comprises:
bringing the cells into contact with an effective amount of the salt according to either of claims 1 and 2 or the p-toluenesulfonate of Compound A according to claim 3 or the pharmaceutical composition according to claim 4.

10. A crystal form of p-toluenesulfonate of Compound A is characterized in that the crystal form is crystal form I, 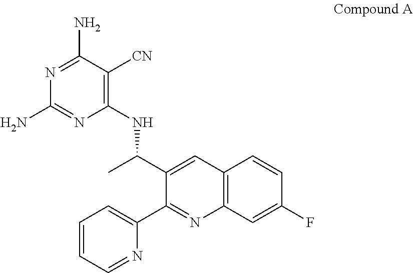 [SMILES: C[C@H](Nc1nc(N)nc(N)c1C#N)c1cc2ccc(F)cc2nc1-c1ccccn1]
the crystal form I has XRPD characteristic peaks at 2θ values of 4.9°±0.2°, 7.6°±0.2°, 12.2°±0.2°, 14.8°±0.2° and 15.4°±0.2°.

11. The crystal form according to claim 10, characterized in that the crystal form I further has at least one XRPD characteristic peak selected from those at 2θ values of 9.8°±0.2°, 10.3°±0.2°, 14.3°±0.2°, 14.5°±0.2°, 16.3°±0.2°, 18.3°±0.2° and 19.8°±0.2°;
preferably, the crystal form I further has at least two XRPD characteristic peaks selected from those at 2θ values of 9.8°±0.2°, 10.3°±0.2°, 14.3° ±0.2°, 14.5°±0.2°, 16.3°±0.2°, 18.3°±0.2°, and 19.8°±0.2°;
preferably, the crystal form I further has at least three XRPD characteristic peaks selected from those at 2θ values of 9.8°±0.2°, 10.3°±0.2°, 14.3°±0.2°, 14.5°±0.2°, 16.3°±0.2°, 18.3°±0.2° and 19.8°±0.2°;
preferably, the crystal form I further has at least four XRPD characteristic peaks selected from those at 2θ values of 9.8°±0.2°, 10.3°±0.2°, 14.3°±0.2°, 14.5°±0.2°, 16.3°±0.2°, 18.3°±0.2° and 19.8°±0.2°;
preferably, the crystal form I further has at least five XRPD characteristic peaks selected from those at 2θ values of 9.8°±0.2°, 10.3°±0.2°, 14.3°±0.2°, 14.5°±0.2°, 16.3°±0.2°, 18.3°±0.2° and 19.8°±0.2°; and
preferably, the crystal form I further has XRPD characteristic peaks at 2θ values of 9.8°±0.2°, 10.3°±0.2°, 14.3°±0.2°, 14.5°±0.2°, 16.3°±0.2°, 18.3°±0.2° and 19.8°±0.2°.

12. The crystal form according to claim 10 or 11, characterized in that the crystal form I has an X-ray powder diffraction pattern essentially as shown in FIG. 13.

13. The crystal form according to claim 10 or 11, characterized in that the crystal form I has a melting peak at 277° C.-283° C.

14. The crystal form according to claim 10 or 11, characterized in that the crystal form I has DSC and TGA thermograms essentially as shown in FIG. 14.

15. A pharmaceutical composition, characterized in that the pharmaceutical composition comprises the crystal form according to any one of claims 10-14 and a pharmaceutically acceptable carrier.

16. Use of the crystal form according to any one of claims 10-14 or the pharmaceutical composition according to claim 15 in the preparation of a medicament for preventing or treating a disease related to phosphoinositide 3-kinase.

17. A method for selectively inhibiting the growth or proliferation of cells containing phosphoinositide 3-kinase in vitro, characterized in that the method comprises:
bringing the cells into contact with an effective amount of the crystal form according to any one of claims 10-14 or the pharmaceutical composition according to claim 15.
